SEQUENCE LISTING (12 sequences)

SEQ ID NO: 1 - DNA, 253 bp, Prevotella
tacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacagg

SEQ ID NO: 2 - DNA, 253 bp, Prevotella
tacgtatggtgcaagcgttatccggatttattgggtttaaagggagcgtaggccggagattaagcgtgttgtgaaatgtagatgctcaacatctgaactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacagg

SEQ ID NO: 3 - DNA, 21 bp, Prevotella
tacggaaggtccgggcgttat

SEQ ID NO: 4 - DNA, 21 bp, Prevotella
agtgcagacgttgagcgtcta

SEQ ID NO: 5 - DNA, 19 bp, Prevotella
tacgtatggtgcaagcgtt

SEQ ID NO: 6 - DNA, 21 bp, Prevotella
gcagttcagatgttgagcatc

SEQ ID NO: 7 - DNA, 252 bp, Prevotella
tacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacag

SEQ ID NO: 8 - DNA, 1494 bp, Prevotella
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaycacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagcctgaaccagccaagtagcgtgcaggatgacggccctatgggttgtaaactgcttttataagggaataaagtgagcctcgtgaggctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaaccttacccgggcttgaattgcagaggaaggatttggagacaatgacgcccttcggggyctctgtgaaggtgctgcatggttgtcgtcagctcgtgccgtgaggtgtcggcttaagtgccataacgagcgcaacccctctccttagttgccatcaggtyaagctgggcactctggggacactgccaccgtaaggtgtgaggaaggtggggatgacgtcaaatcagcacggcccttacgtccggggctacacacgtgttacaatggcaggtacagagagacggtyscyygyaaagtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 9 - DNA, 1491 bp, Prevotella
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaycacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagyctgaaccagccaagtagcgtgcaggawgacggccctatgggttgtaaactgcttttataagggaataaagtgagcctcgtgagrctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctgggggagtacgccggcaacggtgaaactcaaaggaattgacggggcccgcacaagcggaggaacatgtggttaattcgatgatacgcgaggaaccttacccgggcttgaattgcagaggaaggattggagacaatgacgcccttcggggcctctgtgaaggtgctgcatggttgtcgtcagctcgtgccgtgaggtgtcggcttaagtgccataacgagcgcaacccctctccttagttgccatcaggtyawgctgggcactctggggacactgccaccgtaaggtgtgaggaaggtggggatgacgtcaaatcagcayggcccttacgtccggggctacacacgtgttacaatggcaggtacagagagacggtysywygyaarwtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 10 - DNA, 1493 bp, Prevotella
  misc_feature (1038)..(1038): n is a, c, g, or t
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaccacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagyctgaaccagccaagtagcgtgcaggawgacggccctatgggttgtaaactgcttttataagggaataaagtgagcctcgtgagrctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggyctctgtgaangtgctgcatggttgtcgtcagctcgtgccgtgaggtgtcggcttaagtgccataacgagcgcaacccctctccttagttgccatcaggtcaagctgggcactctggggacactgccaccgtaaggtgtgaggaaggtggggatgacgtcaaatcagcacggcccttacgtccggggctacacacgtgttacaatggcaggtacagagagacggtysywtgyaarwtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 11 - DNA, 1493 bp, Prevotella
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaycacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagyctgaaccagccaagtagcgtgcaggatgacggccctatgggttgtaaactgcttttataagggaataaagtgagcctcgtgagrctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggyctctgtgaaggtgctgcatggttgtcgtcagctcgtgccgtgaggtgtcggcttaagtgccataacgagcgcaacccctctccttagttgccatcaggtyaagctgggcactctggggacactgccaccgtaaggtgtgaggaaggtggggatgacgtcaaatcagcacggcccttacgtccggggctacacacgtgttacaatggcaggtacagagagacggtysywygyaarwtsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 12 - DNA, 1493 bp, Prevotella
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaccacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatggrcgagagyctgaaccagccaagtagcgtgcaggawgacggccctatgggttgtaaactgcttttataagggaataaagtgagyctcgtgagrctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggcctctgtgaaggtgctgcatggttgtcgtcagctcgtgccgtgaggtgtcggcttaagtgccataacgagcgcaacccctctccttagttgccatcaggtyaagctgggcactctggggacactgccaccgtaaggtgtgaggaaggtggggatgacgtcaaatcagcacggcccttacgtccggggctacacacgtgttacaatggcaggtacagagagacggtysywtgyaarwwsgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

(19) United States
(12) Patent Application Publication
(10) Pub. No.: US 2021/0177904 A1
(43) Pub. Date: Jun. 17, 2021

(54) BEHAVIOURAL TREATMENT

(71) Applicant: MURDOCH CHILDREN'S RESEARCH INSTITUTE, Parkville, Victoria (AU)

(72) Inventors: Peter VUILLERMIN, Parkville (AU); Anne-Louise PONSONBY, Parkville (AU); Mimi TANG, Parkville (AU); Amy LOUGHMAN, Parkville (AU)

(73) Assignee: MURDOCH CHILDREN'S RESEARCH INSTITUTE, Parkville, Victoria (AU)

(21) Appl. No.: 17/267,877

(22) PCT Filed: Aug. 20, 2019
(86) PCT No.: PCT/AU2019/050878

§ 371 Date: Feb. 11, 2021

(30) Foreign Application Priority Data
Aug. 20, 2018 (AU) 2018903057

(51) Int. Cl. A61K 35/20; A61K 35/741; A61P 25/22

(52) U.S. Cl. CPC A61K 35/20; A61P 25/22; A61K 35/741

(57) ABSTRACT

The invention relates to the treatment and prevention of problem behaviour by the administration of a composition that includes an effective amount of Prevotella.

### FIELD OF THE INVENTION

The invention relates to assessment, treatment and prevention of problem behaviour, including but not limited, to problem behaviour in infants and preschool children.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

There is growing evidence that the gut microbiome is crucial for normal neurodevelopment via neuronal, hormonal and immunological signalling¹. Rodent studies demonstrate that disruptions to gut microbiota are associated with aberrant hypothalamic-pituitary-adrenal axis stress response, decreased expression of brain derived neurotrophic factor and impaired social behaviour in germ-free mice². There may be a critical period for the influence of gut microbiota on neurodevelopment and behaviour, with parallel maturation of the microbiota and central nervous system³. For example, in humans, the microbiota goes through significant development during the first three years of life, mirroring the key developmental window for synoptogenesis^{4,5}.

Clinical studies show altered gut microbiota in people with established conditions such as autism and schizophrenia⁶; however, gut-brain associations in normal human neurodevelopment remain understudied. One study examining cross-sectional relationships between behaviour and gut microbiota in 77 human infants at age 18-27 months reported an association between phylogenetic diversity and temperamental problems, particularly in boys⁷. The only published prospective study of microbiota composition and a subsequent neurodevelopmental outcome in humans demonstrated an inverse association between phylogenetic alpha diversity at 12 months of age and overall cognitive and language measures at 2 years of age⁸.

Intestinal microbial diversity, composition and function is strongly influenced by dietary intake^{9,10} and in particular the reduced intake of fibre-rich carbohydrates, fruit and vegetables, relative to traditional diets¹¹. There is an established link between the Western diet and adverse behavioural phenotypes and mental health outcomes and in both children and adults, including internalizing disorders^{12,13,14}. This diet-mental health association may be explained, at least in part, by gut microbiota composition and metabolic activity, including reduced production of metabolites such as short-chain fatty acids and other neuroactive microbial products of fermentable fibre metabolism¹⁵.

Some options for prevention or treatment of behaviour phenotypes have focussed on increasing or otherwise modifying microbial diversity. These have generally involved consumption of pre-biotic supplements having defined nutrient and non-nutrient components.

There remains a need for preventing and/or reducing the likelihood of development of problem behaviour in an individual.

### SUMMARY OF THE INVENTION

In one embodiment there is provided a method for minimising the likelihood of development of a behaviour in an individual including administering a composition including bacteria to an individual in whom the likelihood of development of a behaviour is to be minimised, wherein the composition includes an effective amount of Prevotella, thereby minimising the likelihood of development of the behaviour in the individual.

In another embodiment there is provided a method for minimising or decreasing or reducing the likelihood of a problem behaviour in an individual including administering a composition including bacteria to an individual in whom the likelihood of a problem behaviour is to be minimised, or decreased or reduced, wherein the composition includes an effective amount of Prevotella, thereby minimising or decreasing or reducing the likelihood of a problem behaviour in the individual.

In another embodiment there is provided a method for minimising the development of a problem behaviour in an individual including administering a composition including bacteria to an individual in whom the development of a problem behaviour is to be minimised, wherein the composition includes an effective amount of Prevotella, thereby minimising the development of the problem behaviour in the individual.

In another embodiment there is provided a method for minimising a problem behaviour in an individual including administering a composition including bacteria to an individual in whom a problem behaviour is to be minimised, wherein the composition includes an effective amount of Prevotella, thereby minimising the problem behaviour in the individual.

In another embodiment there is provided a composition including bacteria for use in minimising the likelihood of development of a behaviour in an individual, or for minimising the likelihood of a problem behaviour in an individual, or for minimising the development of a problem behaviour in an individual, or for minimising a problem behaviour in an individual wherein the composition includes an effective amount of Prevotella.

In another embodiment there is provided a use of a composition including bacteria in the manufacture of a composition for minimising the likelihood of development of a behaviour in an individual, or for minimising the likelihood of a problem behaviour in an individual, or for minimising the development of a problem behaviour in an individual, or for minimising a problem behaviour in an individual wherein the composition includes an effective amount of Prevotella.

In another embodiment there is provided a composition including an effective amount of Prevotella and a further component that is beneficial to an infant.

In another embodiment there is provided a composition including an effective amount of Prevotella and human milk or human milk product.

In another embodiment there is provided a composition including an effective amount of Prevotella, a dietary fibre, peptide and tryptophan.

In another embodiment there is provided a composition including an effective amount of Prevotella, a dietary fibre, peptide, tryptophan and human milk or human milk product.

In another embodiment there is provided a method for determining the likelihood of development of a behaviour in an individual including:
determining whether a stool sample obtained from an individual for whom the likelihood of development of a behaviour is to be determined includes Prevotella;
determining that the individual has a high likelihood of development of the behaviour where the stool sample does not contain Prevotella;
determining that the individual has a low likelihood of development of the behaviour where the stool sample contains Prevotella.

In another embodiment there is provided a method for minimising the likelihood of development of a behaviour in an individual including the following steps:
a) determining the likelihood of development of a behaviour in an individual according to the above described embodiment;
b) administering a composition including bacteria to the individual where the individual is determined by step a) to have a high likelihood of development of a behaviour, wherein the composition includes an effective amount of Prevotella,
thereby minimising the likelihood of development of the behaviour in the individual.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Number of observed OTUs, Chao 1, Shannon and Simpson indices of alpha diversity of 12-month fecal microbiota of infants with elevated behaviour problems (case) and normative behaviour (non-case) at 2 years of age.

FIG. 2. Volcano plot showing the magnitude (log-fold change) versus evidence (log-odds) of differential normalized abundance of all OTUs at 12 months between case and non-case behavioral groups. Evidence of difference was clearly strongest for OTU41 (of genus Prevotella) and OTU35 (of the Lachnospiraceae family). FIG. 3. Carriage and relative abundance of Prevotella and the unspecified genus from the Lachnospiraceae family in fecal microbiota of 12-month old infants who have elevated behavior problems at age 2 (n=22) versus those with normative range behavior problems (n=179). Horizontal solid lines=95% confidence intervals. Vertical dashed line =range of count values, with parentheses indicating the 95% CI of the median. Fractions represent number of individuals with any Prevotella and Lachnospiraceae carriage detected in each group.

### DETAILED DESCRIPTION OF THE EMBODIMENTS

A. Definitions

‘Prevotella’ as used herein refers to a genus of gram negative anaerobic bacteria of the phylum Bacteroidetes. The normalised abundance of the genus Prevotella was significantly greater at 1 year among infants who were not in the behaviour case group at 2 years compared to infants who were in the behaviour case group at 2 years (p value adjusted for multiple comparison <0.001). With regard to simple comparison of presence versus absence, Prevotella was detected in 44% of non-case infants (79/179), but only 4% (1/22) of case infants. OTU41 comprised 95% of all OTUs identified as belonging to the genus Prevotella. The next most common Prevotella OTU was OTU697 at 1.7%. OTUs 41 and 697 were classified as Prevotella_9, a subgroup of Prevotella containing two described species, Prevotella copri and P. paludivivens. Prevotella 9 species X as used herein refers to a strain of Prevotella identifiable by 97% sequence identity to operational taxonomic unit (OTU) 000041 at the V4 16S rDNA locus, which in turn has 100% sequence identity to Prevotella copri. Prevotella 9 species Y as used herein refers to a Prevotella species identifiable by 97% sequence identity to OTU 000697 at the V4 16S rDNA locus. This has 98% similarity to P. copri but is likely a separate strain or species within Prevotella. Prevotella species X and Prevotella species Y are both classified into the Prevotella 9 subgroup.

A composition conditioned by Prevotella as used herein refers to composition that comprises secretions of Prevotella. The composition is preferably acellular. Such a composition may be the supernatant of a culture of Prevotella from which bacterial cells and fragments have been removed.

A composition of the invention that is ‘formulated for human consumption’ as used herein refers to a composition that (a) contains excipients, diluents or carriers that are generally regarded as safe for consumption by humans and/or (b) does not contain ingredients or components that are unsafe for human consumption.

A ‘further component that is beneficial to an infant’ may refer to a dietary fibre and/or amino acid as described herein.

‘Internalising behaviour’ as used herein refers to inward directed behaviours that are indicative of an individual's psychological and emotional state. Examples of internalising behaviour include depression, anxiety, somatic complaint and self harm.

‘Externalising behaviour’ as used herein refers to outward directed behaviours and are reflected by behaviour toward the physical environment. Examples of internalising behaviour include aggression, violence and hyperactivity.

Generally it is recognised that some individuals may display both internalising and externalizing behaviours, so there may be overlap between these classes of behaviour. ‘Problem behaviour’ as used herein generally refers to internalising behaviour, or externalising behaviour or both.

B. Methods for Determining Likelihood of Developing Problem Behaviour

In one aspect the invention is for determining whether in later life an individual is likely to develop, or likely to have developed a problem behaviour. Accordingly the invention provides a method for determining whether an individual has a high or low likelihood of development of a problem behaviour including:
determining whether a test sample, in the form of a stool sample obtained from an individual for whom the likelihood of development of a behaviour is to be determined includes Prevotella;
determining that the individual has a high likelihood of development of the behaviour where the test sample does not contain Prevotella;
determining that the individual has a low likelihood of development of the behaviour where the test sample contains Prevotella.

In an embodiment the invention provides a method for determining whether an individual will have a problem behaviour in later life including:
determining whether a test sample, in the form of a stool sample obtained from an individual for whom problem behaviour in later life is to be determined includes Prevotella;
determining that the individual has a high likelihood of problem behaviour in later life where the test sample does not contain Prevotella;
determining that the individual has a low likelihood of problem behaviour in later life where the test sample contains Prevotella.

The above described methods may involve comparing a test sample with a control sample containing Prevotella that is obtained from an age-matched individual who does not display problem behaviour at 2 years of age. In more detail, a control sample may be obtained at 12 months of age from an individual who at 2 years has not developed problem behaviour. Thus the method may further include:
obtaining a sample from the individual for whom the likelihood of problem behaviour or of developing problem behaviour is to be determined, thereby forming a test sample;

providing a control describing an amount of Prevotella that is obtained from an age-matched individual who does not display problem behaviour at 2 years of age;

comparing the test sample with the control to assess whether the test sample has an amount of Prevotella 16S rDNA as described in the control;

determining that the individual has a low likelihood of problem behaviour or of developing problem behaviour where the test sample has an amount of Prevotella 16S rDNA described in the control;

determining that the individual has a high likelihood of problem behaviour or of developing problem behaviour where the test sample has an amount of Prevotella 16S rDNA that less than that described in the control.

An individual having an absence of Prevotella 16S rDNA in stool, especially of OTU00041 or SEQ ID No: 1 has a higher likelihood of developing problem behaviour.

The control may be derived from one individual, preferably from a cohort of individuals, at least about 10, 20, 50 or 100 individuals.

The control may be in the form of a data file, or in the form of a biological sample.

The control or test sample may be based on assessment of a stool sample, or on the basis of a biological sample from which the amount of 16S rDNA in the stool can be determined. Preferably the test sample is based on assessment of a stool.

The amount or the presence or the absence of 16S rDNA may be determined by any of the techniques described below under this sub-heading. The technique may measure the amount of 16S rDNA directly, or indirectly by measuring some other parameter, for example cfu of Prevotella isolated from a sample.

The method may comprise the assessment of the presence or absence of 16S ribosomal nucleic acid of Prevotella_9, or Prevotella_9 species X, or Prevotella_9 species Y, or OTU00041 or OTU00697, or of SEQ ID No: 1 or of SEQ ID No: 2.

The control may describe the presence of OTU00041 or SEQ ID No: 1, or OTU000697 or SEQ ID No: 2. The test sample may be assessed to determine presence or absence of OTU00041 or SEQ ID No: 1, or OTU000697 or SEQ ID No: 2.

The method may utilize an oligonucleotide having a sequence shown in SEQ ID No: 3, 4, 5 or 6.

Methods for determining the level or amount of bacteria in a sample are known to those skilled in the art. The presence of bacteria may be identified using microbiological culture techniques, biochemical assays or molecular techniques including, but not limited to, PCR (polymerase chain reaction), nucleic acid hybridisation or sequencing techniques. Alternatively, the method may comprise amplifying a bacterial nucleic acid sequence by a technique such as PCR and cloning and/or sequencing the nucleic acid. Identification of bacteria may also be achieved by sequencing of 16S rDNA, including the use of next-generation high-throughput sequencing technologies.

Bacteria may also be detected using immunological methods. For example, antisera or antibodies cross reactive with a bacteria of the genus Prevotella 9 species X and/or Y may be used in a suitable immunological assay. Immunogical assays include enzyme-linked immunosorbent assay (ELISA), and those that use solid supports such as dip-stick type assays. Such immunological assays may utilise labelled antibodies, including fluorescent, radioactive or chemiluminescent labelled antibodies or dye molecules.

Any suitable technique that allows for the qualitative and/or quantitative detection of a nucleic acid from a bacteria of the genus Prevotella 9 species X and/or Y may be used. Comparison may be made by reference to a standard control, or to a negative control. The nucleic acid may be labelled and hybridised on a gene array, in which case the gene concentration will be directly proportional to the intensity of the radioactive or fluorescent signal generated in the array.

In certain embodiments of the present invention, ribosomal nucleic acid can be used to distinguish and detect bacteria. For example, bacterial ribosomes are comprised of a small and large subunit, each which is further comprised of ribosomal nucleic acid and proteins. A large number of ribosomal nucleic acids have been sequenced, and these are publicly available in various accessible databases. Thus, in one embodiment, bacteria of the genus Prevotella 9 species X and/or Y is detected in a sample by sequencing 16S ribosomal nucleic acid amplicons generated by domain-level PCR reactions amplifying from genomic DNA. Traditionally, sequencing of ribosomal nucleic acids was performed by cloning and Sanger (capillary electrophoresis) sequencing of PCR amplicons. The advent of next-generation sequencing has simplified and increased the sequencing depth for 16S ribosomal nucleic acid sequencing.

Accordingly, a “nucleic-acid-based detection assay” as used herein, is an assay for the detection of a target sequence within a target nucleic acid and utilizing one more oligonucleotides that specifically hybridize to the target sequence.

Unless specifically defined otherwise, all technical and scientific terms used herein shall be taken to have the same meaning as commonly understood by one of ordinary skill in the art (e.g., in microbiology, biochemistry, and immunology).

Unless otherwise indicated, the microbiology, biochemistry, and immunological techniques utilized in the present invention are standard procedures, well known to those skilled in the art. Such techniques are described and explained throughout the literature in sources such as, J, Perbal, A Practical Guide to Molecular Cloning, John Wiley and Sons (1984), J. Sambrook and Russell., Molecular Cloning: A Laboratory Manual, 3rd edn, Cold Spring Harbour Laboratory Press (2001), R. Scopes, Protein Purification—Principals and Practice, 3rd edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

C. Methods of Minimising Likelihood of Problem Behaviour

The invention provides methods for conditioning an individual to minimize the likelihood of development of problem behaviour in the individual, or to minimise the likelihood of a problem behaviour in an individual, or to minimise the development of a problem behaviour in an individual, or to minimise a problem behaviour in an individual. It will be understood that the administration of a composition containing an effective amount of Prevotella to the individual is for the purpose of conditioning the individual, or in other words, for the purpose of preparing the individual so as to minimize the likelihood that the individual might in later life display a problem behaviour. Relevantly, in this embodiment, the administration of a composition containing an effective amount of Prevotella is not for the purpose of treating a disease or condition or ailment of the individual at the time that the composition is administered to the individual.

The invention provides a method for minimizing the likelihood of development of a problem behaviour in an individual, or for minimising the likelihood of a problem behaviour in an individual, or for minimising the development of a problem behaviour in an individual, or for minimising a problem behaviour in an individual.

The methods of the invention comprise the step of administering Prevotella to the individual. The administration of Prevotella to the individual is to minimize the likelihood of development of a problem behaviour in the individual, or to minimise the likelihood of a problem behaviour in an individual, or to minimise the development of a problem behaviour in an individual, or to minimise a problem behaviour in an individual. As exemplified herein, the absence of carriage of Prevotella, as observed by absence of Prevotella in faecal compositions produced at 12 months of age is strongly associated with a higher likelihood of problem behaviour arising in in an individual in later life. It follows that this risk factor is minimised by the administration of Prevotella to the individual.

Thus in one embodiment there is provided a method for minimising the likelihood of development of a problem behaviour in an individual including administering a composition including bacteria to an individual in whom the likelihood of development of a problem behaviour is to be minimised, wherein the composition includes an effective amount of Prevotella, thereby minimising the likelihood of development of the problem behaviour in the individual. Preferably the result of the administration of Prevotella to the individual is to increase the relative abundance of Prevotella in the individual thereby minimising the likelihood of development of the problem behaviour in the individual.

In another embodiment there is provided a method for minimising or decreasing or reducing the likelihood of a problem behaviour in an individual including administering a composition including bacteria to an individual in whom the likelihood of a problem behaviour is to be minimised, or decreased or reduced, wherein the composition includes an effective amount of Prevotella, thereby minimising or decreasing or reducing the likelihood of a problem behaviour in the individual.

In another embodiment there is provided a method for minimising the development of a problem behaviour in an individual including administering a composition including bacteria to an individual in whom the development of a problem behaviour is to be minimised, wherein the composition includes an effective amount of Prevotella, thereby minimising the development of the problem behaviour in the individual.

In another embodiment there is provided a method for minimising a problem behaviour in an individual including administering a composition including bacteria to an individual in whom a problem behaviour is to be minimised, wherein the composition includes an effective amount of Prevotella, thereby minimising the problem behaviour in the individual.

In another embodiment there is provided a composition including bacteria for use by administration to an individual in minimising the likelihood of development of a behaviour in an individual, or in minimising the likelihood of a problem behaviour in an individual, or in minimising the development of a problem behaviour in an individual, or in minimising a problem behaviour in an individual wherein the composition includes an effective amount of Prevotella.

The invention provides a use of Prevotella in an individual to minimize the likelihood of development of problem behaviour in the individual.

The individual may be an individual who has been assessed to determine the likelihood of development of problem behaviour, or otherwise likelihood of problem behaviour in later life, including according to a method under the previous subheading. On the basis of the assessment, the individual is administered Prevotella. The individual may be administered Prevotella irrespective of the assessment outcome.

Where the individual is assessed to determine the likelihood of developing a problem behaviour or likelihood of problem behaviour in later life, it is preferred that the assessment is a consideration of the individual's carriage of Prevotella. The assessment may be to determine whether the individual has an absence of detectable Prevotella in the gut. Alternatively, the assessment may be to determine the relative abundance of Prevotella in the gut. Preferably the assessment is to determine whether the individual has an absence of detectable Prevotella in the gut. The assessment may be on the basis of detection of Prevotella specific polynucleotide sequences in the gut or faeces, for example utilizing nucleic acid detection techniques described herein. In other embodiments, the assessment of the risk factor may be generally on the basis of a consideration discussed above.

In another embodiment, the individual to whom the method is applied has not been assessed to determine the likelihood of problem behaviour. According to this embodiment, the individual or parent or family member or guardian of the individual is unaware of the risk profile for the individual developing problem behaviour. According to the embodiment, the individual is administered Prevotella simply to minimize the risk that would apply should the individual have a high likelihood of developing problem behaviour or having problem behaviour in later life.

Prevotella may be administered to the individual at an age of the individual that enables or allows for the minimisation of a behaviour in the individual that, but for the Prevotella administration, would be observable at 2 to 7 years of age, preferably 2 years of age. Generally the Prevotella composition is administered to the individual before the individual is 3 years old, preferably less than 1 to 2 years, preferably 1 day to 12 months, more preferably 6 to 12 months old. Thus the Prevotella may be administered to an infant thereby preventing or minimising problem behaviour in a toddler, pre-school child, adolescent or adult.

The individual is generally a human individual although the embodiments of the invention may relate to other mammalian species.

In a preferred embodiment there is provided a method for minimising the likelihood of an individual having a problem behaviour at 2 years of age including administering a composition including bacteria to an individual in whom the likelihood of development of a problem behaviour at 2 years of age is to be minimised, wherein the composition is administered to the individual when the individual is of 6 to 12 months of age, and wherein the composition includes an effective amount Prevotella, preferably P. copri, thereby minimising the likelihood of development of the problem behaviour in the individual at 2 years of age. Preferably the result of the administration of Prevotella to the individual is to increase the relative abundance of Prevotella in the individual thereby minimising the likelihood of development of the problem behaviour in the individual at 2 years of age.

In embodiments of the invention, the behaviour is problem behaviour in the form of externalising or internalising behaviour, preferably an internalising behaviour. The internalising behaviour may be a disorder selected from the group consisting of depression, anxiety, somatic complaint and suicide. Internalising behaviour in the form of anxiety may be an anxiety selected from the group consisting of separation anxiety, social anxiety disorder, general anxiety disorder, post-traumatic stress disorder, obsessive compulsive disorder and selective mutism as described in Lui J. et al. 2011 J Psychiatr Ment Health Nurs 18:884-894. Externalising behavioural disorders may include aggression, delinquency and hyperactivity as described in Lui J. et al. 2004 J. Child Adolesc Psychiatr Nurs 17:93-103. In a particularly preferred embodiment, Prevotella, preferably P. copri, or composition comprising same, is given at 6 to 12 months of age to minimise the likelihood of observable an internalising behaviour, preferably general anxiety disorder, or separation anxiety or social anxiety disorder, at 2 years of age.

An individual the subject of the administration, especially an infant, may or may not have problem behaviour at the time of administration of the composition.

An individual the subject of the administration, especially an infant, may or may not produce faeces that contain Prevotella at the time of administration of Prevotella. Preferably, at the time of administration of Prevotella, the infant does not produce faeces that contain Prevotella.

An individual the subject of the administration, especially an infant, may or may not produce faeces that have a higher alpha diversity of colon bacteria at the time of administration of Prevotella. Preferably, at the time of administration of Prevotella, the infant produces faeces having a higher alpha diversity of colon bacteria as compared with the faeces of an infant of comparable age who does not later display problem behaviour at 2 to 7 years of age.

An individual the subject of the administration, especially an infant, may or may not produce faeces having a high amount of Lachnospiraceae at the time of administration of Prevotella. Preferably, at the time of administration of Prevotella, the infant produces faeces having a high amount of Lachnospiraceae as compared with faeces of an infant of comparable age who does not later display problem behaviour at 2 to 7 years of age.

An individual the subject of the administration, especially an infant, may or may not have received antibiotic treatment at the time of administration of the Prevotella. Preferably, at the time of administration of Prevotella, the infant had received antibiotic treatment. More preferably the antibiotic treatment was received at about 0 to 9 months of age and the Prevotella is administered at about 6 to 12 months of age.

In one embodiment the individual may be an offspring of a mother who has an absence of Prevotella.

In the above described embodiments, the bacteria that is administered is of the genus Prevotella. The bacteria may consist of a single strain or species of Prevotella, or may comprise different strains or species of Prevotella. For example, the bacteria may be Prevotella copri only, or a specific strain of P. copri only, or more than one strain of P. copri, or P. copri and another Prevotella species.

In the above described embodiments, the Prevotella that is administered to the individual may comprise, or may consist of Prevotella_9. The Prevotella that is administered to the individual may comprise, or may consist of Prevotella_9 species X. The Prevotella that is administered to the individual may comprise, or may consist of Prevotella_9 species Y. The Prevotella that is administered to the individual may comprise, or may consist of Prevotella copri. The Prevotella that is administered to the individual may comprise, or may consist of bacteria having a 16S rDNA sequence shown in SEQ ID No:1; or bacteria having a 16S rDNA sequence having at least 97% identity, preferably 98% identity, preferably 99% identity with the sequence shown in SEQ ID No:1 or bacteria having a 16S rDNA sequence shown in SEQ ID No: 2; or bacteria having a 16S rDNA sequence having at least 97% identity, preferably 98% identity, preferably 99% identity with the sequence shown in SEQ ID No:2.

In the above described embodiments, the individual may be administered with Prevotella to provide from 1×10⁶ to 1×10¹¹ colony forming units (cfu) of Prevotella per day to the individual.

In one embodiment, the administration may provide for establishment of about 10⁷ to 10¹¹ Prevotella in the gastro-intestinal tract of the individual. In one embodiment the administration may provide for an amount of Prevotella in the gastro-intestinal tract of the individual that is observed in individuals that do not present with problem behaviour, preferably do not present with internalising behaviour, at about 2 to 7 years of age.

The Prevotella may be administered once or twice daily, for example at meal times, or once every 2 or 3 days, or once weekly.

In the above described embodiments, the Prevotella may be provided in the form of a composition. Compositions are described under the following sub-heading.

Prevotella may be provided orally to the individual in the form of a capsule, tablet or like formulation adapted for oral administration. In another embodiment the Prevotella may be provided in the form of a food or beverage. Prevotella can be administered in a variety of ways as long as it there is contact between the Prevotella and the gastro-intestinal tract of the individual, preferably with about 10⁷ to 10¹¹ bacteria.

In a particular preferred embodiment, the Prevotella composition is provided together with human milk.

The invention provides a method for minimizing the likelihood of development of problem behaviour, or for preventing problem behaviour, in an individual (i.e. infant or preschool child), comprising orally administering a composition comprising Prevotella copri, to the individual, wherein the administration provides the individual with about 10⁶ to 10¹¹ cfu per day of P. copri throughout months 6 to 12 of the individual's life, thereby minimizing the likelihood of the individual developing a problem behaviour, or preventing a problem behaviour.

The invention provides a composition comprising Prevotella copri for use by oral administration to an individual to provide the individual with about 10⁶ to 10¹¹ cfu per day of P. copri throughout months 6 to 12 of the individual's life, thereby minimizing the likelihood of the individual developing a problem behaviour, or preventing a problem behaviour.

The invention provides a use of Prevotella copri in the manufacture of a composition for oral administration to an individual to provide the individual with about 10⁶ to 10¹¹ cfu per day of P. copri throughout months 6 to 12 of the individual's life, thereby minimizing the likelihood of the individual developing a problem behaviour, or preventing a problem behaviour.

In another embodiment, there is provided treatment of an adolescent or adult having, or at risk of, an internalising or externalising behaviour, preferably internalising behaviour, comprising administering a composition including bacteria to an adult or adolescent in whom internalising or externalising behaviour, or risk of same is to be minimised, wherein the composition includes an effective amount of Prevotella. In this embodiment, the individual is of adolescent or adult age when the Prevotella is administered. The adult or adolescent may not have internalising or externalising behaviour at the time of administration of the composition, and may not produce faeces that contain Prevotella at the time of administration.

D. Compositions & Manufacture

The invention provides a composition formulated for human consumption and uses thereof, the latter as described under the above sub-heading. Typically the composition does not comprise supernatant from Prevotella culture, or components of culture media for culture of Prevotella, other than water. The composition may be used in an above described method, for example to minimize the likelihood of development of problem behaviour. In one embodiment, the composition formulated for human consumption may comprise, or consist of bacteria that is Prevotella. The composition formulated for human consumption may comprise, or consist of bacteria that is Prevotella_9. The composition formulated for human consumption may comprise, or consist of bacteria that is Prevotella_9 species X. The composition formulated for human consumption may comprise, or consist of bacteria that is Prevotella_9 species Y. The composition formulated for human consumption may comprise, or consist of bacteria that is Prevotella copri. In the aforementioned embodiments, the composition may further include a further ingredient selected from the group consisting of a vitamin, a mineral, a long chain polyunsaturated fatty acid, a non digestible oligosaccharide, or a protein, fat or digestible carbohydrate.

In another embodiment there is provided a composition formulated for human consumption. Typically the composition does not comprise supernatant from bacterial culture, or components of culture media for culture of bacteria, other than water. The composition may be used in an above described method, for example to minimize the likelihood of development of problem behaviour. In one embodiment, the composition formulated for human consumption may comprise, or consist of, bacteria selected from the group consisting of: bacteria having a 16S rDNA sequence shown in SEQ ID No:1; or bacteria having a 16S rDNA sequence having at least 97% identity, preferably 98% identity, preferably 99% identity with the sequence shown in SEQ ID No:1 or bacteria having a 16S rDNA sequence shown in SEQ ID No: 2; or bacteria having a 16S rDNA sequence having at least 97% identity, preferably 98% identity, preferably 99% identity with the sequence shown in SEQ ID No:2. In the aforementioned embodiments, SEQ ID No: 1 or SEQ ID No: 2 may each form a V4 structure of a ribosomal RNA molecule. In the aforementioned embodiments, the composition may further include a further ingredient selected from the group consisting of a vitamin, a mineral, a long chain polyunsaturated fatty acid, a non-digestible oligosaccharide, or a protein, fat or digestible carbohydrate.

In another embodiment there is provided a composition formulated for human consumption. Typically the composition does not comprise supernatant from Prevotella culture, or components of culture media for culture of Prevotella, other than water. The composition may be used in an above described method for example to minimize the likelihood of development of problem behaviour. In one embodiment, the composition formulated for human consumption may comprise, or consist of, bacteria selected from the group consisting of: bacteria having a 16S rDNA sequence shown in SEQ ID No:8; or bacteria having a 16S rDNA sequence having at least 80% identity to the sequence shown in SEQ ID No: 8, provided that the sequence includes V4 16S rDNA sequence shown in SEQ ID No: 7; or bacteria having a 16S rDNA sequence shown in SEQ ID No:9; or bacteria having a 16S rDNA sequence having at least 80% identity to the sequence shown in SEQ ID No: 9, provided that the sequence includes V4 16S rDNA sequence shown in SEQ ID No: 7; or bacteria having a 16S rDNA sequence shown in SEQ ID No:10; or bacteria having a 16S rDNA sequence having at least 80% identity to the sequence shown in SEQ ID No: 10, provided that the sequence includes V4 16S rDNA sequence shown in SEQ ID No: 7; or bacteria having a 16S rDNA sequence shown in SEQ ID No:11; or bacteria having a 16S rDNA sequence having at least 80% identity to the sequence shown in SEQ ID No: 11, provided that the sequence includes V4 16S rDNA sequence shown in SEQ ID No: 7; or bacteria having a 16S rDNA sequence shown in SEQ ID No:12; or bacteria having a 16S rDNA sequence having at least 80% identity to the sequence shown in SEQ ID No: 12, provided that the sequence includes V4 16S rDNA sequence shown in SEQ ID No: 7. In the aforementioned embodiments, bacteria may be a strain having a 16S rDNA sequence having at least 85%, or 90%, or 95%, or 96%, or 97%, or 98%, or 99% identity to the sequence shown in any one of SEQ ID No: 8, 9, 10, 11, or 12, provided that the sequence includes SEQ ID No: 7. In the aforementioned embodiments, the composition may further include a further ingredient beneficial for an infant to minimise the likelihood of development of problem behaviour. These further ingredients are described in more detail below.

In another embodiment there is provided a composition formulated for human consumption comprising a strain of Prevotella copri that has been isolated from anaerobic culture medium supernatant, the strain having a 16S rDNA sequence having at least 50% identity to the sequence shown in SEQ ID No: 8 provided that the sequence includes SEQ ID No: 7.

In the above described compositions, bacteria may be a strain having a 16S rDNA sequence having at least 85%, or 90%, or 95%, or 96%, or 97%, or 98%, or 99% identity to the sequence shown in any one of SEQ ID No: 8, 9, 10, 11, or 12, provided that the sequence includes SEQ ID No: 7.

In the above described embodiments, the composition may further include a further ingredient beneficial for an infant to minimise the likelihood of development of problem behaviour.

Percent sequence identity may be determined by conventional methods, by means of computer programs known in the art such as GAP provided in the GCG program package (Program Manual for the Wisconsin Package, Version 8, August 1994, Genetics Computer Group, 575 Science Drive, Madison, Wis., USA 53711) as disclosed in Needleman, S. B. and Wunsch, C D., (1970), Journal of Molecular Biology, 48, 443-453, which is hereby incorporated by reference in its entirety. GAP may be used with the following settings for polynucleotide sequence comparison: GAP creation penalty of 5.0 and GAP extension penalty of 0.3.

In another embodiment there is provided a composition formulated for human consumption comprising a strain of bacteria deposited as DSM number 18205 (JCM13464; CB7) with Leibniz Institute DSMZ-German Collection of Microorganisms and Cell Cultures, Inhoffenstraße 7B 38124 Braunschweig GERMANY. The composition may further comprise a further ingredient beneficial for an infant to minimise the likelihood of development of problem behaviour.

In embodiments herein, reference to a bacterial strain specified by DSM number 18205 may be taken to encompass variants thereof having at least 80% identity with the 16S rRNA sequence of said specified strain, preferably at least 85% identity, more preferably at least 90% identity, further preferably at least 95% identity. In a particularly preferred embodiment, said variant has at least 97% identity with the 16S rRNA sequence of said specified strain, more preferably at least 98% identity, more preferably at least 99% identity.

In embodiments herein reference to Prevotella copri or DSM number 18205 may be taken to include functionally equivalent bacteria derived therefrom such as but not limited to mutants, variants or genetically transformed bacteria. These mutants or genetically transformed strains can be strains wherein one or more endogenous gene(s) of the parent strain has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain to add one or more gene(s) of interest, for instance in order to give to said genetically transformed strains additional physiological features, or to allow them to express proteins of therapeutic or prophylactic interest that one wishes to administer through said strains. These mutants or genetically transformed strains can be obtained from the parent strain by means of conventional techniques for random or site-directed mutagenesis and genetic transformation of bacteria, or by means of the technique known as “genome shuffling”.

The Prevotella utilised in the embodiments of the invention for minimising likelihood of problem behaviour described herein may or may not be antibiotic resistant. Preferably the Prevotella is antibiotic resistant.

In one embodiment there is provided a composition comprising:
Prevotella; and
a further ingredient that is beneficial for an infant to minimise the likelihood of development of problem behaviour, the further ingredient selected from the group consisting of a dietary fibre, a peptide having 7 or more amino acids and a tryptophan source. The dietary fibre may comprise fibres selected from the group of unmodified or modified cereal fibres or modified or unmodified rice fibres and comprise from 15 to 90% wt% non-acetogenic saccharide units. The fibre may be formed of insoluble fibre molecules. The fibre may include a xylan, cellulose, hem i-cellulose, beta 1-3 glucan, lignin and arabinoxylan.

In one embodiment there is provided a composition comprising: Prevotella, a dietary fibre, a peptide having 7 or more amino acids and a tryptophan source. The composition may be used for minimising or treating problem behaviour such as internalizing or externalizing behaviour in a method as described above.

The Prevotella-containing composition may comprise dead bacteria, or live bacteria or a mixture of dead and live bacteria. Preferably at least some of the Prevotella comprised in the composition are living bacteria. Where provided in the form of a unit dose composition (such as a tablet or capsule) a unit dose may have from about 1×10⁶ to 1×10¹² cfu of Prevotella. In other embodiments, a single dosage unit may provide less than 1×10⁶ to 1×10¹² cfu of Prevotella, in which case, 2 or more dosage units are required to provide 1×10⁶ to 1×10¹² cfu of Prevotella.

In one embodiment there is provided a composition conditioned by a bacteria described above, preferably by Prevotella copri bacteria or by a bacteria having a 16S rDNA sequence shown in SEQ ID No:1; or by a bacteria having a 16S rDNA sequence having at least 97% identity, preferably 98% identity, preferably 99% identity with the sequence shown in SEQ ID No:1, or by a bacteria having a 16S rDNA sequence shown in SEQ ID No: 2, or by a bacteria having a 16S rDNA sequence having at least 97% identity, preferably 98% identity, preferably 99% identity with the sequence shown in SEQ ID No:2. The composition may be used in the above described method for minimizing the likelihood of development of behaviour. In one embodiment the composition is acellular. In these embodiments, the composition may further include a further ingredient beneficial for an infant to minimise the likelihood of development of problem behaviour.

In the above described embodiments, the composition may be provided in the form of a capsule, tablet, bead or powder or in the form of a food product.

In the above described embodiments where the composition comprises Prevotella, especially Prevotella copri, the Prevotella cells may be dried, may be microencapsulated, may be coated with an enteric coating (as described below), having an enteric coating.

In the above described embodiments where the composition comprises Prevotella, especially Prevotella copri, the composition may be provided in a form enabling re-hydration before administration.

In one embodiment, the composition may further comprise a desiccant.

In one embodiment, the composition may further comprise an osmoprotectant.

In one embodiment, the composition may further comprise a cryoprotectant.

The invention provides a use of Prevotella in the manufacture of a composition for administration to an individual to minimize the likelihood of development of problem behaviour in the individual.

Prevotella copri may be produced by culture of a strain of bacteria deposited as DSM number 18205 described above.

Alternatively, Prevotella copri may be isolated from feces and the isolate used to produce Prevotella copri cells in culture. Briefly, 0.5 g of a faecal sample is immediately suspended in dilution buffer and 50 ml 10⁸-diluted faecal sample are plated anaerobically on medium 0.05% glucose, 0.05% cellobiose, 0.05% soluble starch, 3.75% minerals, 0.025% L-cysteine HCl.H₂O, 0.0001% resazurin, 0.4% Na₂CO₃, 0.2% trypticase, 0.05% yeast extract, 0.31% volatile fatty acid, 0.001% heroin, and 2.0% agar. Isolates are subcultured on Eggerth Gagnon (EG) agar supplemented with 5% (v/v) horse blood. Isolates that contain Prevotella copri are identified on the basis of containing a 16S nucleotide sequence common to Prevotella_9 species X as described herein. Preferably the isolate contains OTU000041, more preferably, a nucleic acid having SEQ ID No:1 or SE ID No:2 nucleotide sequence. In this embodiment, it is preferred that the Prevotella copri is isolated from the feces or stool of an individual who contains Prevotella copri nucleic acid in his or her stool and who does not have problem behaviour. Such an individual may be identified by the methods of the invention described above.

Prevotella copri strain, whether isolated from faeces, or otherwise may be cultured in 100% CO2 at 37° C. in EG media supplemented with horse blood. Alternatively, cells may be cultured in Columbia blood medium supplemented with 5% defibrinated sheep blood, or PYG medium (modified). The cells may be cultured to an optical density consistent with end of a logarithmic growth phase.

Prevotella including Prevotella derived from a culture method described above may be dried before, during or at completion of formulation. This may improve viability of Prevotella by reducing the water activity of the cells as well as improving the viability and stability of formulations that contain the cells. Prevotella may be dried so as to decrease the water or moisture content of Prevotella cells to about 1 to 10%, preferably from about 2 to 8%, more preferably from about 2 to 5%. Below 1% there may be reduction in cell viability over long term storage. Above 10% the water activity may be too high resulting in reduction in cell viability.

Prevotella may be dried by freeze drying (lyophilization), low temperature vacuum drying (LTVD)or spray drying, or combination of these techniques.

Freeze drying may involve freezing the liquid material in the Prevotella cells with further decreases of the chamber pressure enabling frozen water to sublimate. The key advantage is that the drying step is less damaging than techniques that use higher drying temperatures.

A cryoprotectant may be used in a drying process described above such as freeze drying where the purpose is to freeze water in the cells. The purpose the cryoprotectant is to maintain the viability of the cells as the temperature approaches 0° C. or below. This is achieved by lowering the freezing point of water and consequently its vapour pressure. Examples of cryoprotectants include those that are food grade and those that may permeate through the cell wall. Poly-alcohols such as glycerin, sorbitol and mannitol are examples of cryoprotectants. Other cryoprotectants include oligosaccharides such as inulin, starches and dextrin. Trehalose may be used with sugar alcohols, glycerol or certain proteins, particularly milk derived proteins as a cryoprotectant.

LTVD is based on the principle of creating a vacuum to decrease the pressure around the Prevotella cells below the vapour pressure of water, which decreases the boiling point of water inside the cells. This condition increases the rate of evaporation of water from cells at a temperature that is lower than would otherwise apply if the desired evaporation rate was to be obtained at standard atmospheric condition. One advantage of LVTD is that temperatures at which ice might form in cells are not reached, so this lessens the likelihood of damage to Prevotella cells.

Spray drying is an atomization technology whereby a drying chamber receives a liquid spray containing Prevotella cells which is rapidly evaporated as soon as it encounters a hot air flow producing finely dried particles. Spray drying may involve the use of a stream which acts as an osmoprotectant to protect the Prevotella cells from over drying. These osmoprotectants may include trehalose, non fat milk solids, or adonitol. The osmoprotectants may encapsulate the Prevotella cells as described further below.

During or after drying, Prevotella cells may be microencapsulated. Microencapsulation of Prevotella cells may assist in maintaining the viability of cells during or after drying. Further, microencapsulation may assist in improving stability during storage of Prevotella cells or during passage through the gastro-intestinal tract. Where microencapsulation occurs during drying, the drying step may serve the dual purposes of reducing water content of cells and forming the structure of the microcapsule.

Microencapsulation may take the form of monocore encapsulation in which each capsule contains a single cell, or polycore encapsulation in which each capsule contains more than one cell. A further form is a matrix encapsulation in which individual cells are entrapped within a polymeric material, examples of which include sugars, polysaccharides, proteins and combinations thereof. Monocore or polycore encapsulated Prevotella cells may be entrapped within a polymeric matrix. A microcapsule may have a diameter in the range from 1 micron (monocore encapsulation) through to 1 mm (matrix encapsulation).

Carbohydrate based encapsulates may be used during a freeze drying or spray drying process. Alginate is a common microencapsulation material due to it being nontoxic, relatively cheap and its use in creating matrix microencapsulation in the form of beads. Calcium and sodium alginate are the most widely used forms. Poly-1-lysine alginate composition has also been used for microencapsulation. Alginates are generally used at less than 5% by weight.

Alginate may be combined with cryoprotectants such as glycerol where freeze drying is involved in production, or where Prevotella cells are frozen during storage after drying and encapsulation. Other compounds that may be used with alginate to improve survival include antioxidants (such as ascorbic acid) and buffering agents (phosphate containing buffers).

Polysaccharides such as cellulose acetate phthalate, maltodextrin and modified waxy maize starch have been used as microencapsulants, as have low molecular weight sugars (lactose, trehalose, maltose, and sucrose) and poly-alcohols (mannitol and sorbitol).

The carbohydrate used for microencapsulation may be a prebiotic i.e. a compound or composition that provides growth enhancing effects, or is a nutrient for Prevotella cells in the gastro-intestinal tract.

Protein based encapsulates may also be used during a preparative process for drying cells. These include skim milk, casein and whey protein or non-milk proteins. Plant based proteins such as soy protein has also been used.

Prevotella cells normally inhabit the GI tract in healthy human individuals, including the stomach and duodenal and ileal regions of the small intestine. Endogenous Prevotella therefore has an ability to survive in low pH and bile containing environs.

The stability of cells in the gastro-intestinal tract may be enhanced or improved by using a micro-encapsulant that can either minimize exposure to the environment, particularly so as to provide cells with sufficient time to acclimatize to the environment. For example, a microcapsule may gradually expose cells to low pH or bile over a predetermined time period thereby minimizing the likelihood of inducing shock in the cells. For this purpose, cells may be provided in the form of a monocore or polycore microcapsule, or as a matrix microcapsule. Anyone of these forms of microcapsule may be further provided with a coating in the form of a layer located on or about the microcapsule form to assist in maintenance of viability of cells. The coated microcapsule may be provided in the form of a tablet, a capsule or a bead suitable for oral administration.

Enteric coats are often pH selective and allow for protection against gastric pH and that subsequently dissolve in the more alkali intestinal environment. Various forms of hydroxypropyl methylcellulose (HPMC) including HPMC phthalate have been used to protect orally given bacteria. High amylose starch, particularly chemically substituted starch such as carboxymethyl high amylose starch has also been used. The chemical substitution may minimize degradation of starch in the gastric environment, and, being polysaccharide in nature, the starch is quickly dissolved by enzymatic hydrolysis upon reaching the small intestine. Starch has also been combined with chitosan to provide an enteric coating that may substantially resist degradation in the gastric environment and permit release of cells into the intestine or colon.

Compression coatings which erode over time in gastric and intestinal conditions may be utilized. For example a gel layer of alginate may be used alone or combined with other layers of coating formed from chitosan, whey protein, poly-L-Lysine. Alginate may be mixed with glycerol and xanthan gum. Other microencapsulation systems may use milk protein matrices that are induced by rennet, whey proteins, casein and lactoglobulin.

Generally, Prevotella cells are stored at room temperature, or 4-7° C., or from 0 to 20° C., depending on prior processing and duration of storage.

Cells are generally dried according to a technique described above before storage. If microencapsulation is implemented, it may be necessary to further dry encapsulated cells so as to remove residual water introduced during encapsulation. Removal of water could be carried out through treatments such as use of a desiccant.

Freeze or spray dried microcapsules having low water activity may be stored for no longer than about 8 to 12 weeks without significant impact on viability. Storage for up to 20 months may be possible at colder temperatures from −20 to 7° C.

The viability of dried and/or encapsulated cells can be determined by counting numbers of colony forming units on media described above. This may involve serial diluting from stock derived from a particular batch of Prevotella.

Generally it is preferred that Prevotella composition should have sufficient numbers of cells to provide from 10⁸ to 10⁹ cells to the gastro intestinal tract. Therefore where provided in the form of a unit dose composition (such as a tablet or capsule) a unit dose may have from about 1×10⁶ to 1×10¹¹ cfu of Prevotella.

The invention will now be described with reference to the following, non-limiting examples.

It will be understood that the invention disclosed and defined in this specification extends to all alternative combinations of two or more individual features mentioned or evident from the text or drawings. All of these different combinations constitute various alternative aspects of the invention.

### EXAMPLES

### Example 1

### Association Between Infant Gut Microbiota During Infancy and Problem Behaviour at 2 Years Old

METHOD

A. Sample and Study Design

The Barwon Infant Study (BIS) is a longitudinal, Australian birth cohort study assembled using an unselected antenatal sampling frame¹⁶ (n=1074 infants). Sociodemographic, family history and maternal health data were collected from pregnancy forwards, and faecal samples were collected from infants at 1, 6 and 12 months of age. The 16S rRNA gene was sequenced in a random subsample (n=324), of whom n=201 had all relevant data available and were included in the present study. Parents completed the Child Behavior Checklist¹⁷ (CBCL) when children were 2 years old.

B. Microbiota

B.1 Stool Sample Collection and Processing

Stool samples were collected from BIS infants at 1, 6 and 12 months of age and stored at -80C. In a random subsample of n=201 from the BIS sample, 16S preparation and sequencing was conducted. DNA was extracted using the Qiagen PowerSoil® DNA Isolation Kit, Cat# 12888-100 and transported to the J. Craig Venter Institute, Rockville, Md, USA. The V4 region of the 16S rRNA gene was sequenced on the IIlumina MiSeq platform. USEARCH software was used to merge corresponding paired-end reads, filter (to remove merged reads with mismatches, too many or too few base pairs), cluster into OTUs at 97% identity, identify OUT representative sequences and remove chimeras. The mothur software suite was used to assign representative sequences to taxa described in the SILVA v123 Nr99 taxonomic database. The final descriptions of OTUs present in each sample were composed in USEARCH. Samples with fewer than 2500 read pairs were excluded from further analysis. Fecal samples were transported frozen at −80 C to the CSIRO laboratories, Adelaide, Australia, where short-chain fatty acids were quantified by capillary gas chromatography (GC; 5890 series II Hewlett Packard, Australia.)

C. Behavioural Measurement and Case Definition

The CBCL is a validated 99-item screening questionnaire consisting of problem items within Internalising, Externalising and Total Problems subscales¹⁷. Whilst T scores on these subscales are continuous, the tool provides the following cut-off scores T>64: borderline-clinical range and T>69: clinical range.

In the present study, the cut-off of T>=60 on any subscale (1 standard deviation above the mean) was used, since there was otherwise prohibitively low case ascertainment in this Australian community sample (7/246 for borderline-clinical range; 4/246 for clinical range). Suitability of this approach has been demonstrated in a longitudinal study of conversion to psychiatric disorders diagnosed later in childhood, which showed high specificity (88-96%) and moderate sensitivity (25-34%) with a cut-off of T>6018. The use of a cut-off score at T>70 was found to be too stringent, identifying only 8-20% of those who went on to have psychiatric diagnoses.

Infant temperament at ages 1, 6 and 12 months was measured by parent report using a 5-point likert scale¹⁹.

D. Statistical Analysis

Statistical analysis was conducted in the statistical software environment R, using the Phyloseq package for microbiome data management, the vegan package for beta-diversity and the voom method from the limma package for differential abundance testing. Voom leverages the data from technical replicates to improve statistical efficiency and performs better than competing methods (e.g. DESeq2) for highly variable library sizes, which are typical in 16S studies²⁰. For these voom analyses, we corrected p-values for multiple testing using the Benjamini-Hochberg method and refer to values so corrected as q-values. As the SILVA taxonomy divides accepted genera into multiple clades (e.g. Tyzzerella_x, where x can be 3 or 4), we generated an intermediate taxonomic level between the SILVA family and genus levels by truncating the genus name before its first underscore. This has a secondary effect of consolidating genus level groupings of unnamed clades within families, e.g. Ruminococcaceae_UCG-xxx where xxx can be 013, 014, 010 etc. Logistic and linear regression and chi square tests were used to test for associations between linear and categorical variables as appropriate, including adjusted analyses of the association between the presence of specific bacteria and the behavioural outcome.

E. Microbial Diversity Analyses

Alpha diversity within samples was computed as the Shannon, Simpson, Chao1 and Observed species indices in phyloseq. The effect of alpha diversity and potential confounding variables (determined via the covariate selection process described below) on the binary behavioural outcome was examined via logistic regression.

Beta diversity between samples was computed as the unweighted UniFrac distance²¹. The association between beta diversity and the binary behavioural outcome was examined via the PERMANOVA test; PERMDISP2 was used to examine whether the findings of PERMANOVA could have arisen from differing dispersion between groups.

F. Covariate Adjustment

Analyses of differential abundance were adjusted for the methodological variables: (i) duration of storage of faecal samples in the home freezer (approximately minus 20 degrees) prior to delivery to the laboratory for aliquoting and storage at −80 C, and (ii) duration of storage at −80 C.

Covariates for further adjustment were determined on the basis of a directed acyclic graph procedure as follows. Approximately twenty candidate covariates of relevance to infant microbiota or early childhood behaviour were identified a priori on the basis of theoretical and previously demonstrated relationships (see list below) and entered into a directed acyclic graph (DAG) using DAGitty v3.0. For each of these constructs, a number of candidate variables available in the BIS dataset were specified. Correlation matrices were constructed to inspect linear relationships between candidate variables, candidate microbial exposures and behavioural outcomes of interest. For each construct, the one with the strongest associative relationships with exposures and/or outcomes was selected and entered into the directed acyclic graph. Unmeasured variables were removed. The testable implications from the DAG were analysed using the dagitty package for R. The DAG was updated on the basis of these analyses: new lines were drawn between any associations that failed the tests of independence. Minimal sufficient adjustment sets for estimating the total effect of the microbial exposure on the behavioural outcome were then generated from the web-based software DAGitty.

Results

A. Descriptive statistics of study subcohort

Descriptive characteristics of the full inception cohort (n=1074), the full random subcohort (n=324) and the random subgroup with adequate outcome data at age 2 reported on in the present study (n=201) are presented in Table 1. The cohorts were similar with respect to all relevant characteristics. Twenty-two of the 201 participants for whom all relevant data were available were classified as ‘cases’ on the basis of ‘elevated’ behavioural problems (T>=60; n=14 Externalising subscale, n=9 Internalising subscale, n=10 Total Problems subscale).

B. Covariates Resulting from DAG Process

From the DAG process described above, the a priori DAG was altered and seventeen sets of minimum adjustment sets were obtained. The set of covariates with the most non-missing data was selected: gestational age, mode of birth, antibiotic use during labour, breastfeeding at 4 weeks, number of siblings, household pet ownership. Gender, and child's age in months at time of questionnaire completion were added to this set.

C. Sequencing Summary

Samples from four time-points were sequenced across three sequencing runs. A small number of biological and technical duplicates were sequenced. Among the 12 month samples, all replicates of the same sample clustered together according to weighted UniFrac distance.

E. Child Microbiota and Behavioural Outcomes

D.1 Genus-Level Analyses of 1 and 6mth Microbiota

There was no evidence of associations between 1 or 6 month microbiota alpha diversity (as measured by Shannon Index) or beta diversity (weighted and unweighted UniFrac distances), or differential expression of bacterial genera and the behavioural outcome at age 2 (all p and q>0.05).

D.2 Genus-Level Analyses of 12mth Microbiota

D.2.1 Alpha and Beta Diversity

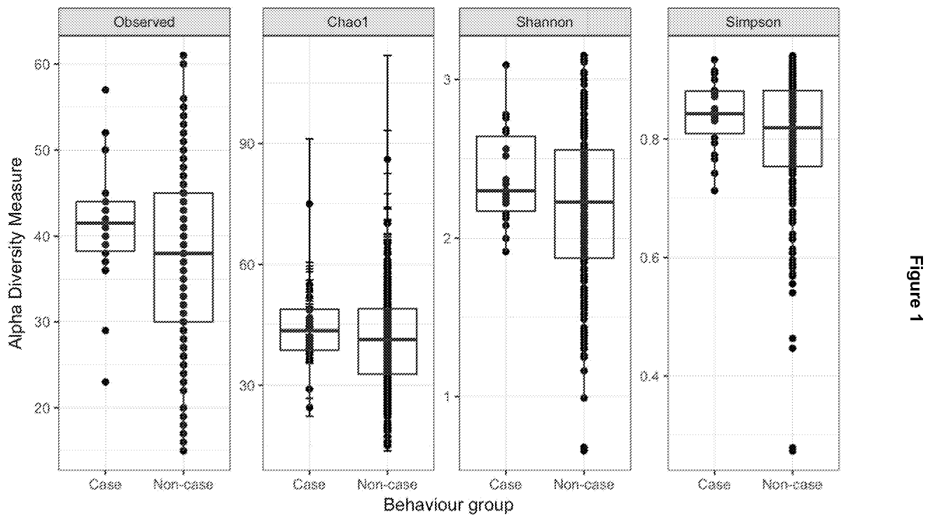

There was weak evidence that greater alpha diversity at 12 months was associated with increased risk of elevated behaviour problems at 2 years, as measured by the Shannon Index (OR: 2.42, 95% CI: 0.92-6.97, p=0.087). A similar pattern was observed using alternative measures of alpha diversity, which also showed greater diversity in the case group (see FIG. 1). Duration of storage was the only one of the processing variables or potentially confounding variables that caused a change of >10% to the estimate of the odds ratio. The evidence of association was minimally changed by adjustment for duration of storage (p=0.07). PERMANOVA applied to unweighted UniFrac distances suggested microbiota community structure differences in the behavioural groups are present (p=0.026) but PERMDISP2 indicated that this may be an artifact of differential multivariate dispersions (p=0.006). There were no observed differences in weighted UniFrac distance.

D.2.2 Differential Abundance

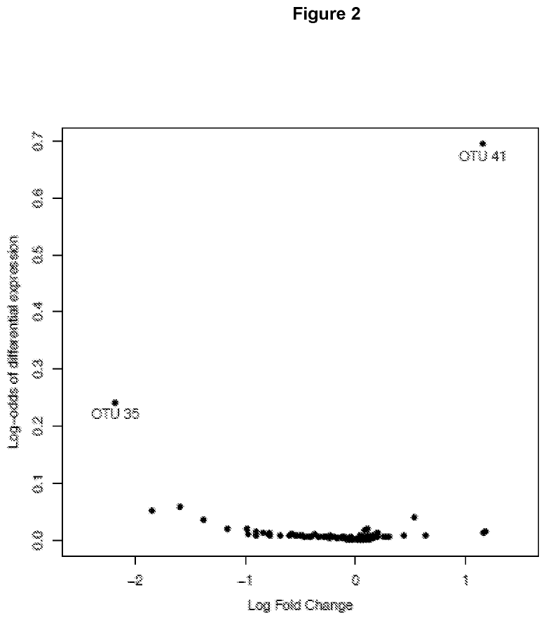

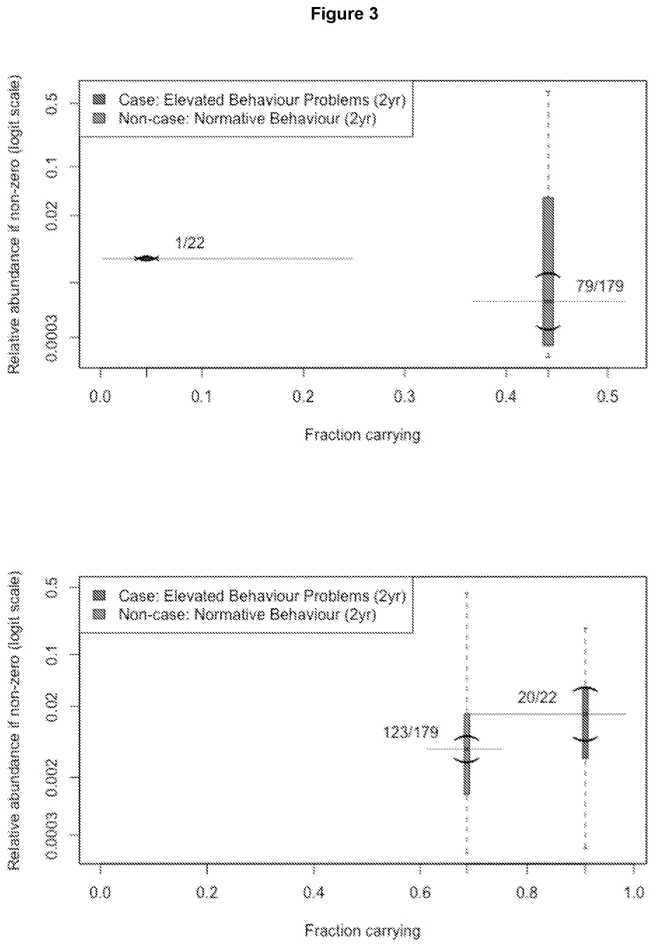

The normalized abundance of two bacterial genera were substantially different in the 12 month faecal microbiota behavioural case infants versus non-case infants: Prevotella (log fold-change (logFC)=1.46, p<0.001, q<0.001) and the collected unspecified genera of the Lachnospiraceae family (logFC=−2.09, p<0.001, q=0.054; see FIG. 2). With regard to simple comparison of presence versus absence, Prevotella was detected in 44% of non-case infants (79/179), but only 4% (1/22) of case infants (FIG. 3). On the other hand, the unspecified Lachnospiraceae genus was detected in 91° A of case infants and 69% of non-case infants (FIG. 3). Table 2 shows the differential abundance of OTUs on the basis of behavioural case status up to q<0.2 (unadjusted analysis).

D.2.3 Investigation of Potential Confounding

Evidence of differential normalized abundance persisted following adjustment for both methodological variables (Prevotella p<0.001, q=0.03; Lachnospiraceae p<0.001, q=0.03) and the potential confounding variables identified via the DAG process: gestational age, mode of birth, antibiotic use during labour, breastfeeding at four weeks, number of older siblings, household pet ownership; Prevotella (p<0.001, q<0.001), Lachnospiraceae (p=0.002, q=0.1). Seven participants were excluded from the adjusted analyses due to missing covariate data. The attenuation evident in these adjusted analyses is consistent with that observed in unadjusted analysis with these participants removed (Prevotella: p<0.001, q<0.001; Lachnospiraceae: p<0.001, q=0.17).

In addition, the association between differential abundance of microbiota genera and the behavioural outcome was tested following adjustment for other exposure variables that may have been proxies for unmeasured antecedent confounding factors. The association between Prevotella carriage and behaviour persisted following adjustment for: maternal prenatal smoking, maternal perceived stress or maternal depressive symptoms at child age 2 (adjustment models run one at a time in addition to minimum adjustment set variables; q<0.001 in all cases). For Lachnospiraceae, the evidence of association persisted following adjustment for maternal prenatal smoking (q=0.022), number of older siblings (q=0.02), perceived stress (q=0.027), number of older siblings (q=0.055), but not following adjustment for breastfeeding (q=0.09), maternal depression (q=0.07).

D.2.4 Investigation of Reverse Causation

There were no associations between child temperament at 1, 6 and 12 months and presence or abundance of Prevotella or Lachnospiraceae at 12 months of age. Nor was there any evidence that the association between the carriage of Prevotella and behaviour at 2 years was attenuated by adjusting for infant temperament at 1, 6 or 12 months of age (q<0.001 in all cases). For Lachnospiraceae, q values were attenuated to 0.15, 0.01 and 0.01 respectively (for 1, 6 and 12 month infant temperament).

D.2.5 CBCL Subscales

The association between Prevotella carriage at 12 months and 2 year behaviour outcomes was primarily related to the internalising subscale as measured by dichotomising at 1 SD above the mean or as a continuous score.

D.2.6 Short-Chain Fatty Acids

There was no evidence of an association between the concentration of faecal short-chain fatty acids and child behaviour (butyrate, propionate, acetate, caproate, valerate, isobutyrate, isovalerate, and a sum of butyrate, acetate, propionate and valerate).

F. OTU members of Prevotella genus and Lachnospiraceae family

Among 12 month samples from the random subcohort, OTU41 comprised 95% of all OTUs identified as belonging to the genus Prevotella. The sequence of 253 base pairs characterising OTU41 was 100% identical to base pairs 529-781 of the P. copri strain JCM 13464 16S rRNA gene (Accession No: AB649279). The next most common Prevotella OTU was OTU697 at 1.7%. The sequence characterising OTU697 differed from that of OTU41 by only 8 base pairs (96.8% identity). OTU697 was only evident in samples in which OTU41 was identified, leading us to believe that this OTU arises from sequencing errors in reads otherwise destined to be classified as OTU41.

The group of Lachnospiraceae OTUs was mostly composed of two OTUs, OTU35 and OTU70 at 56% and 22% respectively, both classified to the Lachnospiraceae NK4A136 group. A BLAST search for the representative sequences of these two OTUs was inconclusive. The group represents 5% of all family Lachnospiraceae OTUs counted in twelve-month samples.

G. Predictors of 12mth Prevotella and Lachnospiraceae Carriage

Antibiotic use from 9 to 12 months (in the 3 months leading up to the faecal sample collection) was associated with reduced presence of Prevotella (OR 0.79 [0.36-1.69], p=0.007), but not Lachnospiraceae (OR 0.67 [0.34-1.33], p=0.24). This association was not observed for antibiotic use earlier in infancy (0-1 month, 1-3 months, 3-6 months, or 6-9 months; all p>0.05). There was no evidence of an association between number of older siblings and carriage of Prevotella at 12 months.

H. 1 and 6mth Prevotella Carriage

Given the strength of association between Prevotella carriage at 12 months and the behavioural outcome, the associations at earlier time-points were assessed. Fewer infants carried Prevotella at 1 or 6 months than at 12 months of age (3% and 24% respectively compared to 40%), and there was no difference in the carriage or abundance of Prevotella at the two earlier time-points by behavioural outcome at 2 years.

Discussion

Our findings indicate a clear association between microbiota composition at 12 months and subsequent child behaviour at 2 years. In particular, increased carriage of the genus Prevotella was associated with decreased internalising behaviours. This association was independent of a range of potential confounding factors. The absence of Prevotella was most clearly predicted by recent exposure to antibiotics.

Prevotella is a gram-negative bacterial genus that appears to be more abundant among populations living in non Westernised environments. It is highly abundant in children in Burkina Faso, and much less abundant in European children, presumably due to vastly different dietary intake of plant polysaccharides, which provide important cellulose and xylans substrates for Prevotella 9. Prevotella dominance has been described as a feature of one so-called ‘enterotypes’, however the notion of enterotypes is controversial, and it has been suggested that consideration of a continuous Bacteroides-Prevotella dominance is a more appropriate metric²². Altered Prevotella abundance has been associated with food-allergy²³ (increased abundance), autism²⁴ (reduced abundance) and Parkinson's disease²⁵ (low abundance had 86.1% sensitivity to the clinical phenotype).

In addition to dietary predictors of Prevotella carriage, high rates of antibiotic-use in modern Western societies, such as that observed in the present study, may also be a relevant consideration. Estimates regarding the duration of disruption to the gut microbiota on the basis of antibiotics vary widely, and are likely to depend on the nature of the antibiotic agent²⁶. Here, only antibiotic exposure at age 9 months—but not earlier—predicted the lower carriage of Prevotella at 12 months, suggesting that Prevotella and other bacteria may repopulate over intervals longer than 3 months.

The potential mechanisms by which Prevotella may influence behaviour are not well established, however may include the common pathways serving gut microbiota and brain communication: the vagus nerve, release of cytokines, tryptophan metabolism, interaction with the peripheral immune system and the production of short-chain fatty acids²⁷. Gut microbial composition and function can act on each of these, which have the potential to affect aspects of neurodevelopment and in turn, the development of appropriate and adaptive behaviour. Although as yet undemonstrated in children, one example from a study of women demonstrated positive associations between abundance of Prevotella, complexity of frontal cortex and insula connections as measured by functional magnetic resonance imaging, and reduced emotional responses to distressing stimuli²⁸. In the present study, the only potential mediating factor measured was the abundance of faecal short-chain fatty acids, and no relationships were observed between any of these and the child behaviour outcome.

There was relatively weaker evidence for two other findings in this study: increased alpha diversity and carriage of Lachnospiraceae in those with subsequent behavioural problems. Reduced microbial diversity is typically associated with poor health outcomes as well as with so-called ‘microbiota depleting’ exposures in modern Western civilisation, such as birth by caesarean section, highly processed dietary intake and high rates of antibiotic use²⁹. However, the current study is concordant with recent reports in a human sample of the same age showing an inverse relationship between 12-month microbiota diversity and 2-year neurodevelopmental (cognitive) outcomes⁸. Microbial diversity is a limited, albeit commonly reported measure of microbial composition in adults, and is commonly believed to represent a ‘healthy’ microbiota profile; however, these studies together suggest that higher microbial diversity in early life may not be favourable for neurodevelopment.

We observed a weak positive association between Lachnospiraceae carriage at 12 months and adverse behavioural outcomes at age 2. Lachnospiraceae is a family of anaerobic bacteria from the order Clostridiales. Some members of this family are fibre fermenters and produce butyrate, a bacterial metabolite shown to have positive neurological effects on tight junction proteins of the blood brain barrier and to reduce neuroinflammation¹⁵. In support of the clinical relevance of Lachnospiraceae family bacteria to neurobiological function, it has also been reported to occur in reduced abundance in faecal microbiota of people experiencing depression³⁰ and is negatively associated with duration of Parkinson's disease³¹. Findings from the present study indicate a potential risk, rather than protective role of Lachnospiraceae, which may reflect the diverse species and their varying functions within this family of bacteria. Further, the association between Lachnospiraceae carriage and the behavioural outcome may have been attributable to confounding since the effect size was significantly attenuated following adjustment.

A strength of this study is that it is prospective and demonstrates a temporal sequence of microbiota exposure and behavioural outcome that is compatible with a causal role of the microbiota. The only previous study to have measured behaviour and the microbiota in human infants did so concurrently⁷. In the present study, there was no evidence of a concurrent association between child temperament at 12 months of age and gut microbiota, making reverse causality or bidirectional explanations of the prospective association unlikely. There was also no apparent association between the normalised abundance of Prevotella, or any other bacteria, in infant gut microbiota at 1 or 6 months and the 2 year behavioural outcome. It is noted that the development of the infant gut microbiota is characterised by rapid and significant change, prompted in particular by changes to feeding and the introduction of solid foods³². The relative lack of diversity, and low rate of carriage of Prevotella at 1 and 6 months may have contributed to the null effect in the earlier two measurements of the infant gut microbiota.

### Example 2

### Minimising Anxiety in Elevated Mouse Model

1. Week 1—Acclimatization:

C57131/6 wild type mice are sorted at the upper animal house into groups of 4 per cage and not handled for the week but closely monitored.

2. Week 2—Treatment with antibiotics:

An antibiotic cocktail (1g/L metronidazole, ampicillin, and neomycin) is given in drinking water for 7 days for a significant depletion or altogether absence of bacteria (Bongers et al., 2014; Rakoff-Nahoum et al., 2004; Scher et al., 2013).

3. Week 3—Bacterial inoculum preparation and P. Copri inoculation.

4. Week 3-7—P. Copri colonization

5. Week 8—Sucrose Preference Test

Animals are given free access to either 1% sucrose solution or normal water overnight. The bottles are weighed at the start and end of a 17-hour period. As mice normally prefer sweet solutions over plain water, impaired preference for sucrose is a well-established indicator of depressive-like behavior similar to anhedonia in humans.

6. Week 9—Open Field test

This test is done in accordance with AWC SOP 89-2010. Animals are placed in the open field test apparatus, a plexiglass box with 16 squares painted on the floor, and left to explore for 6 minutes. The animals are recorded with a video camera.

7. Week 10—Y-Maze

During the first trial, a mouse is gently placed in the middle of the Y-shaped maze, a 3 arm apparatus with a triangular centre, with one arm closed off. The mice are left to explore the two remaining arms for 6 minutes. The mice are then removed from the maze and returned to the home cage for 30 minutes. On the second trial, all arms are open and the number of re-entries into the novel arm vs the previously explored arms are monitored as a measure of their spatial working memory and willingness to explore new environments.

8. Week 11 (Friday) Light Dark box

A mouse is gently placed into the dark portion of the apparatus, a box with a dark room and a bright light room connected by an opening. The number of entries and time spent in each portion of the box are monitored for 6 minutes. These measures are thought to be indices of anxiety-like behavior.

9. Week 12 (Monday) Forced Swim Test

This test is done in accordance with AWC SOP 90-2010. A mouse is gently placed into a 20cm diameter, 50cm high Perspex cylinder filled with 25 degree water to a depth of 18 cm for 6 minutes. At this depth the animal's tail can reach the bottom of the cylinder to provide support. The animal's behavior is monitored and recorded.

### Example 3

### Preventing Internalising Behaviour in a 2 Year Old

Prevotella or P. copri is administered during infancy, preferably after the introduction of solids. Behavioural outcomes are assessed using the Child Behaviour Checklist when the children are two years old. Once the children are school aged, the Spence Children's Anxiety Scale is applied to both the parent and the child to assess anxiety. Comparison is made with a placebo.

Without Limitation the Invention may be Summarised by the Following Items
1. A method for minimising or reducing:
   the likelihood of development of a problem behaviour in an individual or
   the likelihood of a problem behaviour in an individual, or
   the development of a problem behaviour in an individual, or
   a problem behaviour in an individual,
   the method including administering a composition including bacteria to an individual, wherein the composition includes an effective amount of Prevotella.
2. A composition including bacteria for use in minimising the likelihood of development of a problem behaviour in an individual, or for minimising the likelihood of a problem behaviour in an individual, or for minimising the development of a problem behaviour in an individual, or for minimising a problem behaviour in an individual wherein the composition includes an effective amount of Prevotella.
3. The method or composition of items 1 or 2 wherein the individual is 3 years old, preferably less than 1 to 2 years old, preferably 1 day to 12 months, preferably 6 to 12 months old at the time of administration of the composition.
4. The method of any one of the preceding items wherein administration of the composition minimises the development or appearance of a problem behaviour when the individual is at an age of about 2 to 7 years, preferably 2 years.
5. The method of any one of the preceding items where the behaviour is problem behaviour in the form of an internalising behaviour, preferably an anxiety disorder selected from general anxiety disorder, separation anxiety and social anxiety disorder.
6. The method of any one of the preceding items wherein the individual produces faeces that do not contain Prevotella at the time of the administration.
7. The method of any one of the preceding items wherein the individual has received antibiotic treatment prior to the administration of the composition.
8. The method of any one of the preceding items wherein the composition is administered more than once, preferably weekly from 6 to 12 months of age.
9. The method of any one of the preceding items wherein the composition includes Prevotella copri.
10.A composition including an effective amount of Prevotella and a further component that is beneficial to an infant.
11.A composition including an effective amount of Prevotella and human milk or human milk product.
12.A composition including an effective amount of Prevotella, preferably Prevotella copri, a dietary fibre, peptide and tryptophan.
13.A composition including an effective amount of Prevotella, preferably Prevotella copri, a dietary fibre, peptide, tryptophan and human milk or human milk product.
14.A method of any one of the preceding items wherein the composition is according to any one of items 10 to 13.
15.A method for determining the likelihood of development of a problem behaviour in an individual including:

determining whether a stool sample obtained from an individual for whom the likelihood of development of a problem behaviour is to be determined includes Prevotella;

determining that the individual has a high likelihood of development of the behaviour where the stool sample does not contain Prevotella;

determining that the individual has a low likelihood of development of the behaviour where the stool sample contains Prevotella.

Nucleotide Sequences:

(SEQ ID NO: 1)
TACGGAAGGTCCGGGCGTTATCCGGATTTATTGGGTTTAAAGGGAGCGTAG
GCCGGAGATTAAGCGTGTTGTGAAATGTAGACGCTCAACGTCTGCACTGCA
GCGCGAACTGGTTTCCTTGAGTACGCACAAAGTGGGCGGAATTCGTGGTGT
AGCGGTGAAATGCTTAGATATCACGAAGAACTCCGATTGCGAAGGCAGCTC
ACTGGAGCGCAACTGACGCTGAAGCTCGAAAGTGCGGGTATCGAACAGG
(SEQ ID NO: 2)
TACGTATGGTGCAAGCGTTATCCGGATTTATTGGGTTTAAAGGGAGCGTAG
GCCGGAGATTAAGCGTGTTGTGAAATGTAGATGCTCAACATCTGAACTGCA
GCGCGAACTGGTTTCCTTGAGTACGCACAAAGTGGGCGGAATTCGTGGTGT
AGCGGTGAAATGCTTAGATATCACGAAGAACTCCGATTGCGAAGGCAGCTC
ACTGGAGCGCAACTGACGCTGAAGCTCGAAAGTGCGGGTATCGAACAGG
(SEQ ID NO: 3)
TACGGAAGGTCCGGGCGTTAT
(SEQ ID NO: 4)
AGTGCAGACGTTGAGCGTCTA
(SEQ ID NO: 5)
TACGTATGGTGCAAGCGTT
(SEQ ID NO: 6)
GCAGTTCAGATGTTGAGCATC
(SEQ ID No: 7)
TACGGAAGGTCCGGGCGTTATCCGGATTTATTGGGTTTAAAGGGAGCGTAG
GCCGGAGATTAAGCGTGTTGTGAAATGTAGACGCTCAACGTCTGCACTGCA
GCGCGAACTGGTTTCCTTGAGTACGCACAAAGTGGGCGGAATTCGTGGTGT
AGCGGTGAAATGCTTAGATATCACGAAGAACTCCGATTGCGAAGGCAGCTC
ACTGGAGCGCAACTGACGCTGAAGCTCGAAAGTGCGGGTATCGAACAG
(SEQ ID No: 8)
AGAGTTTGATCCTGGCTCAGGATGAACGCTAGCTACAGGCTTAACACATGC
AAGTCGAGGGGAAACGACATCGAAAGCTTGCTTTTGATGGGCGTCGACCGG
CGCACGGGTGAGTAACGCGTATCCAACCTGCCCAYCACTTGGGGATAACCT
TGCGAAAGTAAGACTAATACCCAATGATATCTCTAGAAGACATCTGAAAGA
GATTAAAGATTTATCGGTGATGGATGGGGATGCGTCTGATTAGCTTGTTGG
CGGGGTAACGGCCCACCAAGGCGACGATCAGTAGGGGTTCTGAGAGGAAGG
TCCCCCACATTGGAACTGAGACACGGTCCAAACTCCTACGGGAGGCAGCAG
TGAGGAATATTGGTCAATGGRCGAGAGCCTGAACCAGCCAAGTAGCGTGCA
GGATGACGGCCCTATGGGTTGTAAACTGCTTTTATAAGGGAATAAAGTGAG
CCTCGTGAGGCTTTTTGCATGTACCTTATGAATAAGGACCGGCTAATTCCG
TGCCAGCAGCCGCGGTAATACGGAAGGTCCGGGCGTTATCCGGATTTATTG
GGTTTAAAGGGAGCGTAGGCCGGAGATTAAGCGTGTTGTGAAATGTAGACG
CTCAACGTCTGCACTGCAGCGCGAACTGGTTTCCTTGAGTACGCACAAAGT
GGGCGGAATTCGTGGTGTAGCGGTGAAATGCTTAGATATCACGAAGAACTC
CGATTGCGAAGGCAGCTCACTGGAGCGCAACTGACGCTGAAGCTCGAAAGT
GCGGGTATCGAACAGGATTAGATACCCTGGTAGTCCGCACGGTAAACGATG
GATGCCCGCTGTTGGTCTGAACAGGTCAGCGGCCAAGCGAAAGCATTAAGC
ATCCCACCTGGGGAGTACGCCGGCAACGGTGAAACTCAAAGGAATTGACGG
GGGCCCGCACAAGCGGAGGAACATGTGGTTTAATTCGATGATACGCGAGGA
AACCTTACCCGGGCTTGAATTGCAGAGGAAGGATTTGGAGACAATGACGCC
CTTCGGGGYCTCTGTGAAGGTGCTGCATGGTTGTCGTCAGCTCGTGCCGTG
AGGTGTCGGCTTAAGTGCCATAACGAGCGCAACCCCTCTCCTTAGTTGCCA
TCAGGTYAAGCTGGGCACTCTGGGGACACTGCCACCGTAAGGTGTGAGGAA
GGTGGGGATGACGTCAAATCAGCACGGCCCTTACGTCCGGGGCTACACACG
TGTTACAATGGCAGGTACAGAGAGACGGTYSCYYGYAAAGTSGATCAAATC
CTTAAAGCCTGTCTCAGTTCGGACTGGGGTCTGCAACCCGACCCCACGAAG
CTGGATTCGCTAGTAATCGCGCATCAGCCATGGCGCGGTGAATACGTTCCC
GGGCCTTGTACACACCGCCCGTCAAGCCATGAAAGCCGGGGGCGCCTAAAG
TCCGTGACCGTAAGGAGCGGCCTAGGGCGAAACTGGTAATTGGGGCTAAGT
CGTAACAAGGTAACC
(SEQ ID No: 9)
AGAGTTTGATCCTGGCTCAGGATGAACGCTAGCTACAGGCTTAACACATGC
AAGTCGAGGGGAAACGACATCGAAAGCTTGCTTTTGATGGGCGTCGACCGG
CGCACGGGTGAGTAACGCGTATCCAACCTGCCCAYCACTTGGGGATAACCT
TGCGAAAGTAAGACTAATACCCAATGATATCTCTAGAAGACATCTGAAAGA
GATTAAAGATTTATCGGTGATGGATGGGGATGCGTCTGATTAGCTTGTTGG
CGGGGTAACGGCCCACCAAGGCGACGATCAGTAGGGGTTCTGAGAGGAAGG
TCCCCCACATTGGAACTGAGACACGGTCCAAACTCCTACGGGAGGCAGCAG
TGAGGAATATTGGTCAATGGRCGAGAGYCTGAACCAGCCAAGTAGCGTGCA
GGAWGACGGCCCTATGGGTTGTAAACTGCTTTTATAAGGGAATAAAGTGAG
CCTCGTGAGRCTTTTTGCATGTACCTTATGAATAAGGACCGGCTAATTCCG
TGCCAGCAGCCGCGGTAATACGGAAGGTCCGGGCGTTATCCGGATTTATTG
GGTTTAAAGGGAGCGTAGGCCGGAGATTAAGCGTGTTGTGAAATGTAGACG
CTCAACGTCTGCACTGCAGCGCGAACTGGTTTCCTTGAGTACGCACAAAGT
GGGCGGAATTCGTGGTGTAGCGGTGAAATGCTTAGATATCACGAAGAACTC
CGATTGCGAAGGCAGCTCACTGGAGCGCAACTGACGCTGAAGCTCGAAAGT
GCGGGTATCGAACAGGATTAGATACCCTGGTAGTCCGCACGGTAAACGATG
GATGCCCGCTGTTGGTCTGAACAGGTCAGCGGCCAAGCGAAAGCATTAAGC
ATCCCACCTGGGGGAGTACGCCGGCAACGGTGAAACTCAAAGGAATTGACG
GGGCCCGCACAAGCGGAGGAACATGTGGTTAATTCGATGATACGCGAGGAA
CCTTACCCGGGCTTGAATTGCAGAGGAAGGATTGGAGACAATGACGCCCTT
CGGGGCCTCTGTGAAGGTGCTGCATGGTTGTCGTCAGCTCGTGCCGTGAGG
TGTCGGCTTAAGTGCCATAACGAGCGCAACCCCTCTCCTTAGTTGCCATCA
GGTYAWGCTGGGCACTCTGGGGACACTGCCACCGTAAGGTGTGAGGAAGGT
GGGGATGACGTCAAATCAGCAYGGCCCTTACGTCCGGGGCTACACACGTGT
TACAATGGCAGGTACAGAGAGACGGTYSYWYGYAARWTSGATCAAATCCTT
AAAGCCTGTCTCAGTTCGGACTGGGGTCTGCAACCCGACCCCACGAAGCTG
GATTCGCTAGTAATCGCGCATCAGCCATGGCGCGGTGAATACGTTCCCGGG
CCTTGTACACACCGCCCGTCAAGCCATGAAAGCCGGGGGCGCCTAAAGTCC
GTGACCGTAAGGAGCGGCCTAGGGCGAAACTGGTAATTGGGGCTAAGTCGT
AACAAGGTAACC
(SEQ ID No: 10)
AGAGTTTGATCCTGGCTCAGGATGAACGCTAGCTACAGGCTTAACACATGC
AAGTCGAGGGGAAACGACATCGAAAGCTTGCTTTTGATGGGCGTCGACCGG
CGCACGGGTGAGTAACGCGTATCCAACCTGCCCACCACTTGGGGATAACCT
TGCGAAAGTAAGACTAATACCCAATGATATCTCTAGAAGACATCTGAAAGA
GATTAAAGATTTATCGGTGATGGATGGGGATGCGTCTGATTAGCTTGTTGG
CGGGGTAACGGCCCACCAAGGCGACGATCAGTAGGGGTTCTGAGAGGAAGG
TCCCCCACATTGGAACTGAGACACGGTCCAAACTCCTACGGGAGGCAGCAG
TGAGGAATATTGGTCAATGGRCGAGAGYCTGAACCAGCCAAGTAGCGTGCA
GGAWGACGGCCCTATGGGTTGTAAACTGCTTTTATAAGGGAATAAAGTGAG
CCTCGTGAGRCTTTTTGCATGTACCTTATGAATAAGGACCGGCTAATTCCG
TGCCAGCAGCCGCGGTAATACGGAAGGTCCGGGCGTTATCCGGATTTATTG
GGTTTAAAGGGAGCGTAGGCCGGAGATTAAGCGTGTTGTGAAATGTAGACG
CTCAACGTCTGCACTGCAGCGCGAACTGGTTTCCTTGAGTACGCACAAAGT
GGGCGGAATTCGTGGTGTAGCGGTGAAATGCTTAGATATCACGAAGAACTC
CGATTGCGAAGGCAGCTCACTGGAGCGCAACTGACGCTGAAGCTCGAAAGT
GCGGGTATCGAACAGGATTAGATACCCTGGTAGTCCGCACGGTAAACGATG
GATGCCCGCTGTTGGTCTGAACAGGTCAGCGGCCAAGCGAAAGCATTAAGC
ATCCCACCTGGGGAGTACGCCGGCAACGGTGAAACTCAAAGGAATTGACGG
GGGCCCGCACAAGCGGAGGAACATGTGGTTTAATTCGATGATACGCGAGGA
ACCTTACCCGGGCTTGAATTGCAGAGGAAGGATTTGGAGACAATGACGCCC
TTCGGGGYCTCTGTGAANGTGCTGCATGGTTGTCGTCAGCTCGTGCCGTGA
GGTGTCGGCTTAAGTGCCATAACGAGCGCAACCCCTCTCCTTAGTTGCCAT
CAGGTCAAGCTGGGCACTCTGGGGACACTGCCACCGTAAGGTGTGAGGAAG
GTGGGGATGACGTCAAATCAGCACGGCCCTTACGTCCGGGGCTACACACGT
GTTACAATGGCAGGTACAGAGAGACGGTYSYWTGYAARWTSGATCAAATCC
TTAAAGCCTGTCTCAGTTCGGACTGGGGTCTGCAACCCGACCCCACGAAGC
TGGATTCGCTAGTAATCGCGCATCAGCCATGGCGCGGTGAATACGTTCCCG
GGCCTTGTACACACCGCCCGTCAAGCCATGAAAGCCGGGGGCGCCTAAAGT
CCGTGACCGTAAGGAGCGGCCTAGGGCGAAACTGGTAATTGGGGCTAAGTC
GTAACAAGGTAACC
(SEQ ID No: 11)
AGAGTTTGATCCTGGCTCAGGATGAACGCTAGCTACAGGCTTAACACATGC
AAGTCGAGGGGAAACGACATCGAAAGCTTGCTTTTGATGGGCGTCGACCGG
CGCACGGGTGAGTAACGCGTATCCAACCTGCCCAYCACTTGGGGATAACCT
TGCGAAAGTAAGACTAATACCCAATGATATCTCTAGAAGACATCTGAAAGA
GATTAAAGATTTATCGGTGATGGATGGGGATGCGTCTGATTAGCTTGTTGG
CGGGGTAACGGCCCACCAAGGCGACGATCAGTAGGGGTTCTGAGAGGAAGG
TCCCCCACATTGGAACTGAGACACGGTCCAAACTCCTACGGGAGGCAGCAG
TGAGGAATATTGGTCAATGGRCGAGAGYCTGAACCAGCCAAGTAGCGTGCA
GGATGACGGCCCTATGGGTTGTAAACTGCTTTTATAAGGGAATAAAGTGAG
CCTCGTGAGRCTTTTTGCATGTACCTTATGAATAAGGACCGGCTAATTCCG
TGCCAGCAGCCGCGGTAATACGGAAGGTCCGGGCGTTATCCGGATTTATTG
GGTTTAAAGGGAGCGTAGGCCGGAGATTAAGCGTGTTGTGAAATGTAGACG
CTCAACGTCTGCACTGCAGCGCGAACTGGTTTCCTTGAGTACGCACAAAGT
GGGCGGAATTCGTGGTGTAGCGGTGAAATGCTTAGATATCACGAAGAACTC
CGATTGCGAAGGCAGCTCACTGGAGCGCAACTGACGCTGAAGCTCGAAAGT
GCGGGTATCGAACAGGATTAGATACCCTGGTAGTCCGCACGGTAAACGATG
GATGCCCGCTGTTGGTCTGAACAGGTCAGCGGCCAAGCGAAAGCATTAAGC
ATCCCACCTGGGGAGTACGCCGGCAACGGTGAAACTCAAAGGAATTGACGG
GGGCCCGCACAAGCGGAGGAACATGTGGTTTAATTCGATGATACGCGAGGA
ACCTTACCCGGGCTTGAATTGCAGAGGAAGGATTTGGAGACAATGACGCCC
TTCGGGGYCTCTGTGAAGGTGCTGCATGGTTGTCGTCAGCTCGTGCCGTGA
GGTGTCGGCTTAAGTGCCATAACGAGCGCAACCCCTCTCCTTAGTTGCCAT
CAGGTYAAGCTGGGCACTCTGGGGACACTGCCACCGTAAGGTGTGAGGAAG
GTGGGGATGACGTCAAATCAGCACGGCCCTTACGTCCGGGGCTACACACGT
GTTACAATGGCAGGTACAGAGAGACGGTYSYWYGYAARWTSGATCAAATCC
TTAAAGCCTGTCTCAGTTCGGACTGGGGTCTGCAACCCGACCCCACGAAGC
TGGATTCGCTAGTAATCGCGCATCAGCCATGGCGCGGTGAATACGTTCCCG
GGCCTTGTACACACCGCCCGTCAAGCCATGAAAGCCGGGGGCGCCTAAAGT
CCGTGACCGTAAGGAGCGGCCTAGGGCGAAACTGGTAATTGGGGCTAAGTC
GTAACAAGGTAACC
(SEQ ID No: 12)
AGAGTTTGATCCTGGCTCAGGATGAACGCTAGCTACAGGCTTAACACATGC
AAGTCGAGGGGAAACGACATCGAAAGCTTGCTTTTGATGGGCGTCGACCGG
CGCACGGGTGAGTAACGCGTATCCAACCTGCCCACCACTTGGGGATAACCT
TGCGAAAGTAAGACTAATACCCAATGATATCTCTAGAAGACATCTGAAAGA
GATTAAAGATTTATCGGTGATGGATGGGGATGCGTCTGATTAGCTTGTTGG
CGGGGTAACGGCCCACCAAGGCGACGATCAGTAGGGGTTCTGAGAGGAAGG
TCCCCCACATTGGAACTGAGACACGGTCCAAACTCCTACGGGAGGCAGCAG
TGAGGAATATTGGTCAATGGRCGAGAGYCTGAACCAGCCAAGTAGCGTGCA
GGAWGACGGCCCTATGGGTTGTAAACTGCTTTTATAAGGGAATAAAGTGAG
YCTCGTGAGRCTTTTTGCATGTACCTTATGAATAAGGACCGGCTAATTCCG
TGCCAGCAGCCGCGGTAATACGGAAGGTCCGGGCGTTATCCGGATTTATTG
GGTTTAAAGGGAGCGTAGGCCGGAGATTAAGCGTGTTGTGAAATGTAGACG
CTCAACGTCTGCACTGCAGCGCGAACTGGTTTCCTTGAGTACGCACAAAGT
GGGCGGAATTCGTGGTGTAGCGGTGAAATGCTTAGATATCACGAAGAACTC
CGATTGCGAAGGCAGCTCACTGGAGCGCAACTGACGCTGAAGCTCGAAAGT
GCGGGTATCGAACAGGATTAGATACCCTGGTAGTCCGCACGGTAAACGATG
GATGCCCGCTGTTGGTCTGAACAGGTCAGCGGCCAAGCGAAAGCATTAAGC
ATCCCACCTGGGGAGTACGCCGGCAACGGTGAAACTCAAAGGAATTGACGG
GGGCCCGCACAAGCGGAGGAACATGTGGTTTAATTCGATGATACGCGAGGA
ACCTTACCCGGGCTTGAATTGCAGAGGAAGGATTTGGAGACAATGACGCCC
TTCGGGGCCTCTGTGAAGGTGCTGCATGGTTGTCGTCAGCTCGTGCCGTGA
GGTGTCGGCTTAAGTGCCATAACGAGCGCAACCCCTCTCCTTAGTTGCCAT
CAGGTYAAGCTGGGCACTCTGGGGACACTGCCACCGTAAGGTGTGAGGAAG
GTGGGGATGACGTCAAATCAGCACGGCCCTTACGTCCGGGGCTACACACGT
GTTACAATGGCAGGTACAGAGAGACGGTYSYWTGYAARWWSGATCAAATCC
TTAAAGCCTGTCTCAGTTCGGACTGGGGTCTGCAACCCGACCCCACGAAGC
TGGATTCGCTAGTAATCGCGCATCAGCCATGGCGCGGTGAATACGTTCCCG
GGCCTTGTACACACCGCCCGTCAAGCCATGAAAGCCGGGGGCGCCTAAAGT
CCGTGACCGTAAGGAGCGGCCTAGGGCGAAACTGGTAATTGGGGCTAAGTC
GTAACAAGGTAACC

TABLE 1
Descriptive characteristics of cohort and subgroup in the current study.

| | | Random |
|---|---|---|
| | Inception | subcohort with |
| | birth cohort | adequate date |
| Characteristic | (n = 1074) | (n = 201) |
| Sex of child: | | |
| Male | 555 (51.7%) | 106 (52.7%) |
| Female | 519 (48.3%) | 95 (47.3%) |
| Twins (sets) | 10 (1%) | 2 (1%) |
| Maternal country of birth | | |
| Australia | 961 (89.5%) | 179 (89.1%) |
| Other | 110 (10.2%) | 21 (10.4%) |
| Unknown | 3 (0.3%) | 1 (0.5%) |
| Paternal country of birth | | |
| Australia | 91.5 (85.2%) | 169 (84.1%) |
| Other | 108 (10.1%) | 19 (9.4%) |
| Unknown | 51 (4.8%) | 13 (6.5%) |
| Maternal age, years (mean & SD) | 31.3 (4.8) | 32.4 (4.2) |
| Paternal age, years (mean & SD) | 33.5 (5.9) | 34.2 (5.1) |
| Maternal education | | |
| <year 10 of high school | 12 (1.1%) | 2 (1.0%) |
| Year 10 of high school | 80 (7.5%) | 14 (7.0%) |
| Year 12 of high school | 162 (15.1%) | 28 (13.9%) |
| Trade, certificate, diploma | 266 (24.8%) | 39 (19.4%) |
| Bachelor degree | 354 (33%) | 76 (37.8%) |
| Postgraduate degree | 194 (18.1%) | 42 (20.1%) |
| Unknown | 6 (.6%) | 0 (0.0%) |
| SEIFA | | |
| Low | 357 (33.2%) | 59 (29.4%) |
| Middle | 353 (32.9%) | 73 (36.3%) |
| High | 351 (32.7%) | 69 (34.3%) |
| unknown | 13 (1.9%) | 0 (0.0%) |
| Number of siblings including twins | | |
| 0 | 473 (44.0%) | 77 (36.3%) |
| 1 | 383 (35.7%) | 87 (44.3%) |
| 2 | 177 (16.5%) | 32 (16.4%) |
| 3 or more | 41 (3.8%) | 5 (2.5%) |
| Maternal cigarette smoking in | | |
| pregnancy: | | |
| Yes | 169 (15.7%) | 19 (9.5%) |
| No | 892 (83.1%) | 182 (90.5%) |
| Unknown | 13 (1.2%) | 0 (0.0%) |
| Pet ownership | | |
| Yes | 790 (73.6%) | 143 (71.1%) |
| No | 278 (25.9%) | 58 (28.9%) |
| Unknown | 6 (0.6%) | 0 (0.0%) |
| Livestock ownership | | |
| Yes | 73 (6.8%) | 10 (5.0%) |
| No | 985 (91.7%) | 188 (93.5%) |
| Unknown | 16 (1.5%) | 3 (1.5%) |
| Delivered in a government hospital | | |
| Yes | 780 (72.6%) | 121 (60.2%) |
| No | 294 (27.4%) | 80 (39.8%) |
| Delivered via Caesarean section | 333 (31%) | 70 (34.9%) |
| Gestational age at birth: | | |
| 32 to 36 completed weeks | 47 (4.4%) | 4 (2%) |
| 37 to 42 completed weeks | 1,027 (95.6%) | 197 (99%) |
| >42 completed weeks | 0 (0.0%) | 0 (0.0%) |
| Birth weight in grams (mean & SD) | 3528 (519) | 3522 (522) |

TABLE 2
Evidence of differential abundance at the family/genus
level on the basis of 2 year behavior group*

| Characteristic | | Log | | |
|---|---|---|---|---|
| OTU ref- | | fold | | |
| erence number | Family/Genus | change | p | q |
| 41 | Prevotella | −1.46 | <0.001 | <0.001 |
| 35 | Lachnospiraceae | 2.09 | <0.001 | 0.05 |
| 7 | Bacteroides | 1.64 | 0.008 | 0.28 |
| 77 | Incertae** | 1.46 | 0.01 | 0.28 |
| 22 | Ruminococcus | 1.70 | 0.02 | 0.28 |
| 1106 | uncultured** | 1.10 | 0.03 | 0.37 |

*Note:
Log fold change indicates change in case infants relative to non-case group; positive values indicate greater expression in the case group, negative values indicate greater expression in the non-case group.
**Both OTUs 77 and 1106 are identified as Lachnospiraceae at the family level.

### REFERENCES

1. Foster J A, Rinaman L, Cryan J F. Stress & the gut-brain axis: Regulation by the microbiome. Neurobiol Stress. 2017; 7:124-136. doi:10.1016/j.ynstr.2017.03.001

2. Sudo N, Chida Y, Aiba Y, et al. Postnatal microbial colonization programs the hypothalamic-pituitary-adrenal system for stress response in mice. J Physiol. 2004; 558(1):263-275. doi:10.1113/jphysio1.2004.063388

3. Tognini P. Gut Microbiota: A Potential Regulator of Neurodevelopment. Front Cell Neurosci. 2017; 11(February):1-8. doi:10.3389/fnce1.2017.00025

4. Tau G Z, Peterson B S. Normal Development of Brain Circuits. Neuropsychopharmacol Rev. 2010; 35115(10):147-168. doi:10.1038/npp.2009.115

5. de Weerth C. Do Bacteria Shape our Development? Crosstalk between Intestinal Microbiota and HPA Axis. Neurosci Biobehav Rev. 2017; (September):1-14. doi:10.1016/j.neubiorev.2017.09.016

6. Kelly J R, Minuto C, Cryan J F, Clarke G, Dinan T G. Cross talk: The microbiota and neurodevelopmental disorders. Front Neurosci. 2017; 11(SEP):1-31. doi:10.3389/fnins. 2017.00490

7. Christian L M, Galley J D, Hade E M, Schoppe-Sullivan S, Kamp Dush C, Bailey M T. Gut microbiome composition is associated with temperament during early childhood. Brain Behav Immun. 2015; 45(2015):118-127. doi:10.1016/j.bbi.2014.10.018

8. Carlson A L, Xia K, Azcarate-Peril M A, et al. Infant Gut Microbiome Associated With Cognitive Development. Biol Psychiatry. 2017. doi:10.1016/j.biopsych.2017.06.021

9. De Filippo C, Cavalieri D, Di Paola M, et al. Impact of diet in shaping gut microbiota revealed by a comparative study in children from Europe and rural Africa. Proc Natl Acad Sci. 2010; 107(33):14691-14696. doi:10.1073/pnas.1005963107

10. Schnorr S L, Candela M, Rampelli S, et al. Gut microbiome of the Hadza hunter-gatherers. Nat Commun. 2014;5:3654.

11. De Filippis F, Pellegrini N, Vannini L, et al. High-level adherence to a Mediterranean diet beneficially impacts the gut microbiota and associated metabolome. Gut. 2016; 65(11): 1812-1821. doi:10.1136/gutjnl-2015-309957

12. Jacka F N, Ystrom E, Brantsaeter A L, et al. Maternal and early postnatal nutrition and mental health of offspring by age 5 years: A prospective cohort study. J Am Acad Child Adolesc Psychiatry. 2013; 52(10):1038-1047. doi:10.1016/j.jaac.2013.07.002

13. Jacka F N, Pasco J A, Mykletun A, et al. Association of Western and traditional diets with depression and anxiety in women. Am J Psychiatry. 2010; 167(3):305-311.

14. Jacka F N, Pasco J A, Mykletun A, et al. Diet quality in bipolar disorder in a population based sample of women. J Affect Disord. 2011; 129(1):332-337.

15. Bourassa M W, Alim I, Bultman S J, Ratan R R. Butyrate, neuroepigenetics and the gut microbiome: Can a high fiber diet improve brain health? Neurosci Lett. 2016; 625:56-63. doi:10.1016/j.neulet.2016.02.009

16. Vuillermin P, Saffery R, Allen K J, et al. Cohort Profile: The Barwon Infant Study. Int J Epidemiol. 2015; 44(4):1148-1160. doi:10.1093/ije/dyv026

17. Achenbach T M. Child Behavior Checklist and related instruments. 1994.

18. Petty C R, Rosenbaum J F, Hirshfeld-Becker D R, et al. The child behavior checklist broadband scales predict subsequent psychopathology: A 5-year follow-up. J Anxiety Disord. 2008; 22(3):532-539. doi:10.1016/j.janxdis.2007.04.003

19. Ponsonby A L, Dwyer T, Couper D. Factors related to infant apnoea and cyanosis: A population-based study. J Paediatr Child Health. 1997; 33(4):317-323. doi:10.1111/j. 1440-1754.1997.tb01608.x

20. Law C W, Chen Y, Shi W, Smyth G K. Voom: Precision weights unlock linear model analysis tools for RNA-seq read counts. Genome Biol. 2014; 15(2). doi:10.1186/gb-2014-15-2-r29

21. Lozupone C, Lladser M E, Knights D, Stombaugh J, Knight R. UniFrac: an effective distance metric for microbial community comparison. ISME J. 2011; 5(2):169.

22. Gorvitovskaia A, Holmes S P, Huse S M. Interpreting Prevotella and Bacteroides as biomarkers of diet and lifestyle. Microbiome. 2016; 4(1):15. doi:10.1186/s40168-016-0160-7

23. Ling Z, Li Z, Liu X, et al. Altered fecal microbiota composition associated with food allergy in infants. Appl Environ Microbiol. 2014; 80(8):2546-2554.

24. Kang D-W, Park J G, Ilhan Z E, et al. Reduced incidence of Prevotella and other fermenters in intestinal microflora of autistic children. PLoS One. 2013; 8(7):e68322.

25. Scheperjans F, Aho V, Pereira PAB, et al. Gut microbiota are related to Parkinson's disease and clinical phenotype. Mov Disord. 2015; 30(3):350-358.

26. Langdon A, Crook N, Dantas G. The effects of antibiotics on the microbiome throughout development and alternative approaches for therapeutic modulation. Genome Med. 2016; 8(1):39. doi:10.1186/s13073-016-0294-z

27. Dinan T G, Cryan J F. Microbes, Immunity, and Behavior: Psychoneuroimmunology Meets the Microbiome. Neuropsychopharmacology. 2017; 42(1):178-192. doi:10.1038/npp. 2016.103

28. Tillisch K, Labus J, Kilpatrick L, et al. Consumption of fermented milk product with probiotic modulates brain activity. Gastroenterology. 2013; 144(7):1394-1401.e4. doi: 10.1053/j.gastro.2013.02.043

29. Mosca A, Leclerc M, Hugot J P. Gut microbiota diversity and human diseases: Should we reintroduce key predators in our ecosystem? Front Microbiol. 2016; 7(MAR):1-12. doi: 10.3389/fmicb.2016.00455

30. Naseribafrouei A, Hestad K, Avershina E, et al. Correlation between the human fecal microbiota and depression. Neurogastroenterol Motil. 2014; 26(8):1155-1162.

31. Trasande L, Zoeller R T, Hass U, et al. Estimating burden and disease costs of exposure to endocrine-disrupting chemicals in the European Union. J Clin Endocrinol Metab. 2015; 100(4):1245-1255. doi:10.1210/jc.2014-4324

32. Koenig J E, Spor A, Scalfone N, et al. Succession of microbial consortia in the developing infant gut microbiome. Proc Natl Acad Sci. 2011; 108(Supplement 1):4578-

1. A method for minimising the likelihood of development of a problem behaviour in an individual including administeting a composition including bacteria to an individual in whom the likelihood of development of a behaviour is to be minimised, wherein the composition includes an effective amount of Prevotella, thereby minimising the likelihood of development of the behaviour in the individual, wherein the composition is administered to the individual before the individual is 3 years old.

2. The method of claim 1 wherein the composition is administered to the individual at an age of the individual that enables minimisation or reduction of a problem behaviour in the individual at 2 to 7 years of age.

3. (canceled)

4. (canceled)

53. The method of claim 1 wherein the behaviour is problem behaviour in the form of externalising or internalising behaviour.

6. The method of claim 5 wherein the internalising behaviour is a disorder selected from the group consisting of depression, anxiety, somatic complaint and suicide.

7. The method of claim 6 wherein the anxiety is selected from the group consisting of separation anxiety, social anxiety disorder, general anxiety disorder, post-traumatic stress disorder, obsessive compulsive disorder and selective mutism.

8. The method of claim 1 wherein the individual does not have problem behaviour at the time of administration of the composition.

9. The method of claim 1 wherein the individual produces faeces that do not contain Prevotella at the time of administration of the corn position.

10. The method of claim 1 wherein the individual produces faeces that have a high alpha diversity of colon bacteria at the time of administration of the composition.

11. The method of claim 1 wherein the individual produces faeces having a high amount of Lachnospiraceae at the time of administration of the composition.

12. The method of claim 1 wherein the individual has received antibiotic treatment.

13. The method of claim 1 wherein the composition includes human milk.

14. The method of claim 1 wherein the composition is administered more than once.

15. The method of claim 1 wherein the composition includes an effective amount of Prevotella copri.

16-20. (canceled)
